(19) United States
(12) Patent Application Publication
(10) Pub. No.: US 2022/0313135 A1
(43) Pub. Date: Oct. 6, 2022

(54) METHOD AND SYSTEM FOR MEASURING EEG SIGNALS

(71) Applicant: EEG-SENSE Ltd., Kiryat Ono (IL)

(72) Inventors: Israel DEUTSCH, Petach-Tikva (IL); Yair LEVY, Moshav Almagor (IL)

(73) Assignee: EEG-SENSE Ltd., Kiryat Ono (IL)

(21) Appl. No.: 17/620,158

(22) PCT Filed: Jun. 18, 2020
(86) PCT No.: PCT/IL2020/050685

§ 371 Date: Dec. 17, 2021

Related U.S. Application Data
(60) Provisional application No. 62/862,689, filed on Jun. 18, 2019.

(51) Int. Cl. A61B 5/291; A61B 5/251; A61B 5/377; A61B 5/00; A61N 1/04; A61N 1/36

(52) U.S. Cl. CPC A61B 5/291; A61B 5/251; A61B 5/377; A61B 5/6803; A61B 5/6843; A61B 5/7221; A61B 5/7455; A61N 1/0456; A61N 1/0476; A61N 1/36034

(57) ABSTRACT

A system for measuring EEG signals, comprises a wearable body adapted to fit over a scalp, a plurality of electrodes mounted on the wearable body, and optionally also a plurality of controllable actuators for applying force to the electrodes. A controller optionally controls each actuator or group of actuators to apply force to at least one electrode. A signal processor receives and processes signals from the electrodes and optionally transmits control signals to the controller based on the processing.

### RELATED APPLICATION

This application claims the benefit of priority of U.S. Provisional Patent Application No. 62/862,689 filed on Jun. 18, 2019, the contents of which are incorporated herein by reference in their entirety.

### FIELD AND BACKGROUND OF THE INVENTION

The present invention, in some embodiments thereof, relates to electroencephalography (EEG) and, more particularly, but not exclusively, to a method and system for measuring EEG signals.

The brain is a complex structure of nerve cells that produce signals called excitatory post synaptic potentials (EPSP). These potentials summate in the cortex and extend through the coverings of the brain to the scalp, where they can be measured using appropriate electrodes. Rhythmical measured activity represents postsynaptic cortical neuronal potentials which are synchronized by the complex interaction of large populations of cortical cells. EEG, a noninvasive recording technique, is one of the commonly used systems for monitoring brain activity. EEG data is simultaneously collected from a multitude of channels at a high temporal resolution, yielding high dimensional data matrices for the representation of brain activity. In addition to its unsurpassed temporal resolution, EEG is non-invasive, wearable, and more affordable than other neuroimaging techniques.

In some techniques, EEG signals are acquired by headsets.

One very common example of an EEG headset used in medical applications is a standard EEG cap, which includes a cap made of a rubber or rubber-like material that is put on the subject's head like a swimming cap. The cap has surface electrodes positioned throughout the cap in a manner such that they come in contact with surface of the subject's head. Common contemporary EEG headsets require extensive preparation along with professional lengthy setup and positioning. In order to improve conductivity, gel or saline is typically employed. In some cases, uncomfortable pressure is applied to the electrodes to enhance the electrical coupling. Also known, is the use of dry electrodes, that allow fast and easy setup. Once an EEG headset is worn, a trial-and-error procedure is employed to ensure that the headset is properly aligned with the desired locations on the scalp and properly attached to the scalp to achieve good conductivity.

### SUMMARY OF THE INVENTION

According to an aspect of some embodiments of the present invention there is provided a system for measuring electroencephalography (EEG) signals. The system comprises a wearable body adapted to fit over a scalp, a plurality of electrodes mounted on the wearable body at a density of at least 2 electrodes per 3 cm², and a signal processor configured for detecting Evoked Related Potential (ERP) signals from the electrodes and determining a physiological location of each electrode or each group of electrodes based on the ERP signals.

According to some embodiments of the invention the system comprises an input for receiving from a stimulation system signals describing stimulation of a subject wearing the wearable body, wherein the signal processor configured for determining the location based in part on the signals from the stimulation system.

According to some embodiments of the invention the stimulation system is configured to apply electrical stimulation by at least one of the electrodes.

According to an aspect of some embodiments of the present invention there is provided a system for measuring EEG signals. The system comprises: a wearable body adapted to fit over a scalp; a plurality of electrodes mounted on the wearable body; a plurality of stretch sensors for generating stretch data describing a stretching of the wearable body; and a signal processor configured for receiving the stretch data and constructing from the stretch data a three-dimensional map describing physiological locations of the electrodes over the wearable body, once stretched.

According to some embodiments of the invention the system comprises a plurality of physically separate sensing systems, each comprising several of the plurality of electrodes.

According to an aspect of some embodiments of the present invention there is provided a system for measuring EEG signals. The system comprises: a wearable body adapted to fit over a scalp; a plurality of electrodes mounted on the wearable body; a plurality of controllable actuators for applying force to the electrodes; a controller configured for individually controlling each actuator or group of actuators to apply force to at least one electrode; and a signal processor configured for receiving and processing signals from the electrodes and transmitting control signals to the controller based on the processing.

According to some embodiments of the invention the signal processor is configured for determining at least one of: an electrode-tissue impedance, a signal-to-noise ratio, artifacts percentage, and a signal quality, and to control the force based on the determination.

According to some embodiments of the invention at least one of the electrodes is flexible and configured to experience a strain once pressed by the force against the scalp.

According to some embodiments of the invention the actuator is configured to apply the force while establishing rotary motion to the electrodes.

According to some embodiments of the invention at least one of the controllable actuators comprises an inflatable balloon, applying the force upon inflation thereof.

According to some embodiments of the invention the force is periodic and is applied to vibrate the electrodes or generate a hammering effect.

According to some embodiments of the invention the system comprises a plurality of physically separate sensing systems, each comprising several of the plurality of electrodes.

According to some embodiments of the invention at least one of the sensing systems comprises a circuit board and a plurality of flexible legs, each having a non-conductive section and a conductive section having a tip in electrical communication with the circuit board, wherein each conductive section is one of the plurality of electrodes.

According to some embodiments of the invention the circuit board and the plurality of flexible legs are detachable from each other.

According to some embodiments of the invention the wearable body comprises an inner shell supporting the circuit board and the plurality of flexible legs, and an outer shell supporting the plurality of controllable actuators.

According to some embodiments of the invention at least one of the flexible legs has a helical shape.

According to some embodiments of the invention a conductive section of at least one of the plurality of legs is polymeric.

According to some embodiments of the invention a conductive section of at least one of the plurality of legs comprises a bundle of conductive bristles.

According to some embodiments of the invention the system comprises a controllable vibrating member configured for vibrating the legs.

According to some embodiments of the invention at least one of the legs comprises a hydrophobic zone at an upper part of the leg and a hydrophilic zone at a lower part of the leg.

According to some embodiments of the invention the plurality of flexible legs is arranged on a base of a sensing system body, wherein the sensing system(s) comprises a shaft and a housing mounted on the shaft and being configured to receive the sensing system body, and wherein the housing comprises a rigid wall for holding the sensing system body and a flexible membrane connecting the rigid wall with the shaft in a manner that allows the housing to assume a plurality of different orientations with respect to the shaft.

According to some embodiments of the invention the system comprises a controller for controlling a connection state of each individual electrode.

According to some embodiments of the invention the system comprises a wired bus interface for establishing communication between the controller and the electrodes.

According to some embodiments of the invention the controller is configured for electrically grouping the electrodes into at least one group.

According to some embodiments of the invention the group(s) forms a predetermined morphology over a surface of the scalp.

According to some embodiments of the invention the group(s) is selected in a closed loop control based on at least one of: an electrode-tissue impedance, a signal-to-noise ratio, artifacts percentage and a signal quality, calculated by the signal processor for signals received by the electrodes.

According to an aspect of some embodiments of the present invention there is provided a method of measuring EEG signals. The method comprises operating the system according to any of claims 1-22, while the wearable body is placed on a scalp of a subject, to receive EEG signals sensed by the plurality of electrodes, thereby measuring the EEG signals.

According to an aspect of some embodiments of the present invention there is provided a system for sensing electroencephalography signals. The system comprises a circuit board and a plurality of flexible legs, each having a non-conductive section and a conductive section having a tip in electrical communication with the circuit board, wherein at least one of the flexible legs has a helical shape.

According to an aspect of some embodiments of the present invention there is provided a system for sensing electroencephalography signals. The system comprises a circuit board and a plurality of flexible legs, each having a non-conductive section and a conductive section having a tip in electrical communication with the circuit board, wherein a conductive section of at least one of the plurality of legs comprises a bundle of conductive bristles.

According to an aspect of some embodiments of the present invention there is provided a system for sensing electroencephalography signals. The system comprises a circuit board and a plurality of flexible legs, each having a non-conductive section and a conductive section having a tip in electrical communication with the circuit board, wherein at least one of the legs comprises a hydrophobic zone at an upper part of the leg and a hydrophilic zone at a lower part of the leg.

According to some embodiments of the invention the leg(s) comprises an intermediate zone between the hydrophobic zone and the hydrophilic zone, the intermediate zone being less hydrophobic than the hydrophobic zone, and less hydrophilic than the hydrophilic zone.

According to an aspect of some embodiments of the present invention there is provided a method of measuring EEG signals. The method comprises: detecting Evoked Response Potential (ERP) signals from a plurality of electrodes placed on a surface scalp of a subject at a density of at least 2 electrodes per 3 cm²; and processing the ERP to determine a physiological location of each electrode or each group of electrodes on the surface based on the ERP signals.

According to some embodiments of the invention the method comprises receiving from a stimulation system signals input describing stimulation of the subject, wherein the determination of the location is based in part on the signals from the stimulation system.

According to an aspect of some embodiments of the present invention there is provided a method of measuring EEG signals. The method comprises: receiving signals from a plurality of electrodes placed on a surface scalp of a subject; and processing the signals and controlling a plurality of controllable actuators to apply force to the electrodes, based on the processing.

According to some embodiments of the invention the method comprises determining at least one quantity selected from the group consisting of: an electrode-tissue impedance, a signal-to-noise ratio, an artifacts percentage, and a signal quality, and increasing the force responsively to a value of the at least one quantity.

According to some embodiments of the invention the force is applied inwardly.

According to some embodiments of the invention at least one of the controllable actuators comprises an inflatable balloon, wherein the controlling comprising inflating the balloon to apply the force.

According to some embodiments of the invention the method comprises determining a pressure in the balloon and at least one quantity selected from the group consisting of: an electrode-tissue impedance, a signal-to-noise ratio, an artifacts percentage, and a signal quality, and, varying the pressure responsively to a value of the at least one quantity.

According to some embodiments of the invention the controlling comprises operating the actuators to apply the force periodically so as to vibrate the electrodes or generate a hammering effect.

According to some embodiments of the invention the method comprises determining at least one quantity selected from the group consisting of: an electrode-tissue impedance, a signal-to-noise ratio, an artifacts percentage, and a signal quality, and operating the actuators to vibrate the electrodes or generate the hammering effect responsively to a value of the at least one quantity.

According to some embodiments of the invention the method comprises varying a connection state of at least one of the electrodes.

According to some embodiments of the invention the method comprises electrically grouping the electrodes into at least one group.

According to some embodiments of the invention the method comprises selecting the group(s) in accordance with a predetermined morphology over a surface of the scalp.

According to some embodiments of the invention the method comprises determining at least one quantity selected from the group consisting of: an electrode-tissue impedance, a signal-to-noise ratio, an artifacts percentage, and a signal quality, and selecting the group(s) in a closed loop control based on a value of the quantity(ies).

According to an aspect of some embodiments of the present invention there is provided a jig system for collectively assembling a plurality of sensing systems, each sensing system being configured for sensing EEG signals. The jig system comprises a scaffold having an outer surface and an inner surface, the outer surface being designed and constructed to fittedly receive a wearable body mounted with a plurality of housings; a plurality of recesses formed in the outer surface, each having a base and a through hole formed between the base and the inner surface of the scaffold, each recesses being size-wise and shape-wise compatible with a disposable electrode assembly of one of the sensing systems; and a plurality of jig shafts, each introduced into one of the recesses from a side of the inner surface via a respective through holes.

According to an aspect of some embodiments of the present invention there is provided a kit. The kit comprises the jig system as delineated above and optionally and preferably as further detailed below; and a system for measuring EEG signals, wherein the system comprises a wearable body adapted to fit over a scalp, and a plurality of housings mounted on the wearable body, each housing having therein a circuit board of one of the sensing systems, and being configured to receive an electrode assembly of one of the sensing systems.

According to some embodiments of the invention the kit comprises a plurality of disposable electrode assemblies each being size-wise and shape-wise compatible with one of the recesses.

According to an aspect of some embodiments of the present invention there is provided a method of assembling a system for measuring EEG signals. The method comprises placing a plurality of disposable electrode assemblies of a respective plurality of sensing systems in a respective plurality of recesses of a jig system such as, but not limited to, the jig system delineated above and optionally and preferably as further detailed below; mounting on the scaffold a wearable body having thereon a plurality of housings each being configured to receive one of the electrode assemblies, and having therein a circuit board of one of the sensing systems, so as to align the housings with the recesses; and activating the jig shafts to release the electrode assemblies out of recesses and to connect the electrode assemblies to the housings.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A and 1B are schematic illustrations of a system for measuring EEG signals, according to some embodiments of the present invention;

FIG. 1C is a schematic illustration showing a mounting configuration of electrodes on a wearable body according to some embodiments of the present invention;

FIGS. 1D-F are schematic illustrations showing representative examples of shapes suitable for the electrodes shown in FIG. 1C, according to some embodiments of the present invention;

FIGS. 2A-E are schematic illustrations of a wearable body which comprises a plurality of stretch sensors, according to some embodiments of the present invention;

FIGS. 3A-F are schematic illustrations of a sensing system that comprises several electrodes, in embodiments of the invention in which metallic wires are employed;

FIGS. 4A-F are schematic illustrations of a sensing system that comprises several electrodes, in embodiments of the invention in which a bundle of conductive bristles is employed;

FIGS. 5A-F are schematic illustrations of a sensing system that comprises several electrodes, in embodiments of the invention in which a bundle of a conductive polymer is employed;

FIG. 6 is a schematic illustration of a helical sensing leg, according to some embodiments of the present invention;

FIG. 7 is a schematic illustration of a sensing leg in embodiments of the invention in which the leg comprises a hydrophobic zone and a hydrophilic zone;

FIGS. 8A-H are schematic illustrations of a system a system for measuring EEG signals in embodiments of the invention in which the system comprises controllable actuators for applying force to the electrodes;

FIGS. 9A and 9B are schematic illustrations of the system in embodiments of the invention in which the actuator comprises a balloon, a shaft, and a stator, and the wearable body comprises an outer shell and an inner shell;

FIG. 9C is a schematic illustration of a sheet member and a plurality of bodies of sensing systems embedded therein, according to some embodiments of the present invention;

FIGS. 10A and 10B are schematic illustrations describing a bus interface according to some embodiments of the present invention;

FIG. 11 is a flowchart diagram describing a close loop procedure for operating an actuator to apply a compression force on an electrode, according to some embodiments of the present invention;

FIG. 12 is a flowchart diagram describing a close loop procedure for operating an actuator to vibrate the electrodes, according to some embodiments of the present invention;

FIG. 13 is a flowchart diagram describing a close loop procedure for operating an actuator to provide a hammering effect, according to some embodiments of the present invention;

FIGS. 14A-F are schematic illustrations showing additional configurations of a sensing leg according to some embodiments of the present invention;

FIG. 15 is a flowchart diagram describing a combined procedure for operating an actuator, according to some embodiments of the present invention; and

FIGS. 16A-D which are schematic illustrations of a jig system according to some embodiments of the present invention.

### DESCRIPTION OF SPECIFIC EMBODIMENTS OF THE INVENTION

The present invention, in some embodiments thereof, relates to EEG and, more particularly, but not exclusively, to a method and system for measuring EEG signals.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The inventors realized that there are several technological challenges in a measurement of EEG signals. EEG signals can be collected using wet electrodes or dry electrodes. It was realized by the inventors that using wet electrodes requires time consuming preparations, skilled experienced operators and complicates the process, because a conductive gel should be applied on the scalp, and because subject's preparation oftentimes requires hair shaving and removal of dead skin from the scalp. The gel may also cause discomfort to the subject, and may also result in skin irritation. Additionally, since the gel is drying over time, it needs to be reapplied regularly (typically every hour). Further, the amount of applied gel needs to be considered with great care, since too much gel may cause short-circuits between electrodes, and to less gel may result in poor conductivity.

It was realized by the inventors that the use of dry electrodes, is not without certain operative limitations that would best be avoided. These include the need to manually place the electrodes one by one and to apply pressure on the electrodes so as to ensure penetration through the hair and good contact. The applied pressure may also cause discomfort since the subject may suffer from constant tension on the scalp. Use of dry electrode may also cause discomfort due to entanglement between the electrodes and the hair.

Furthermore, known EEG systems include a wired cable per electrode. This limits the ability of the subject to move, and may also reduce the signal quality since unavoidable movements of the subject may result in signal disruptions (artifacts).

Additionally, in traditional EEG systems, there is a requirement for accurate placement of each electrode on a specific location over the scalp (e.g., according to the International 10-20 EEG scheme). The Inventors realized that deviations from the specific locations can reduce the accuracy of the diagnostics.

In a search for a solution to the above problems the Inventors devised a technique for sensing and measuring EEG signals. The technique of the present embodiments can be used to investigate and/or diagnose various conditions and disorders, including, without limitation, seizures, traumatic brain injury (TBI), hemorrhages, brain tumors, encephalopathy, cognitive decline, sleep disorders, ischemic pathologies, e.g., stroke, dementia and coma level of brain activity. The technique of the present embodiments can be used to evaluate several types of brain disorders. For example, when epilepsy is present, seizure activity can appear as rapid spiking waves on the EEG, and when the brain includes lesions, which can result from tumors or stroke, unusually slow EEG waves can be detected, depending on the size and the location of the lesion. The technique of the present embodiments can be used in biofeedback applications for various purposes, including, without limitation, improving the quality of life for subjects suffering from motor disorders or motor dysfunction.

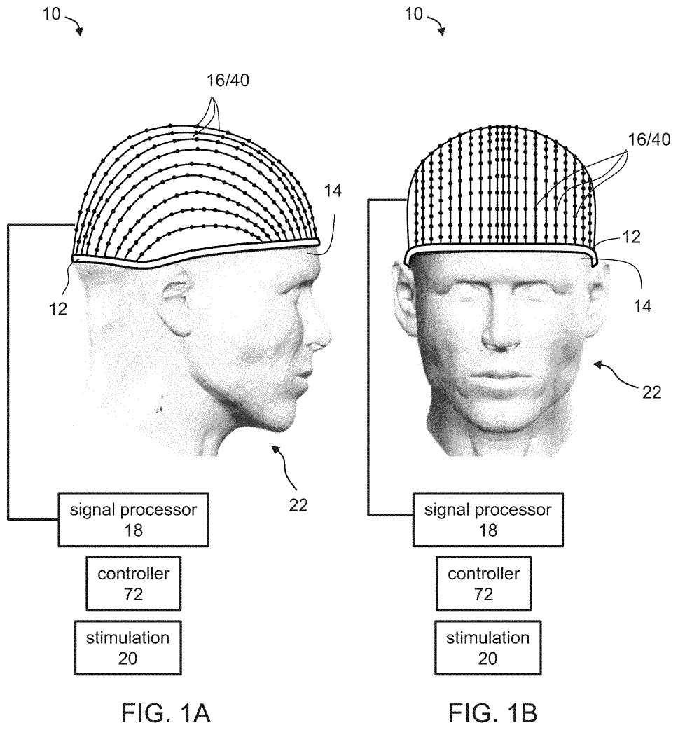

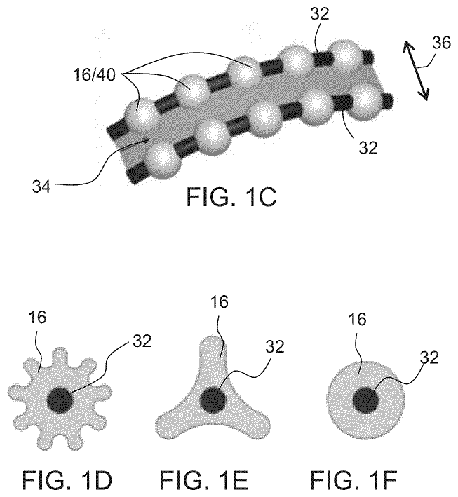

Referring now to the drawings, FIGS. 1A and 1B are schematic illustrations of a system 10 for measuring EEG signals, according to some embodiments of the present invention. In various exemplary embodiments of the invention preferably, but not necessarily, system 10 is a dry EEG system, in the sense that the system can measure EEG signals by establishing contact with a scalp 14 of a subject 22 in dry environment, and in particular without applying gel between the sensing elements and the skin.

System 10 preferably comprises a wearable body 12 adapted to fit over scalp 14. Wearable body 12 can be rigid, flexible, or elastic, and can be made of any material that can be worn over the scalp, such as, but not limited to, a synthetic fabric, molded plastic, fiberglass, reinforced fiberglass (e.g., reinforced with Kevlar or carbon fibers).

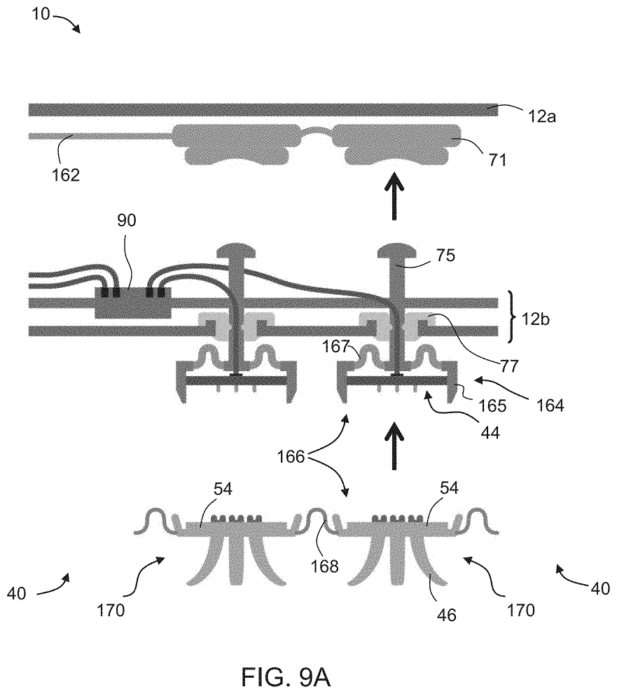

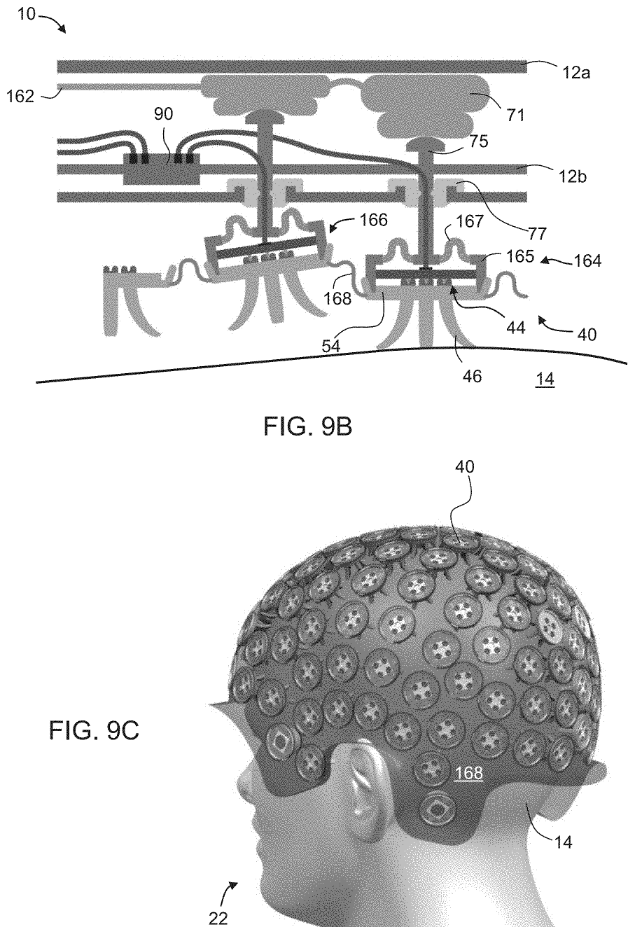

In some embodiments of the present invention wearable body 12 comprises an outer shell and an inner shell (not shown in FIGS. 1A and 1B, see FIGS. 9A and 9B described below). In some embodiments of the present invention at least a portion of wearable body 12 is elastic. System 10 further comprises a plurality of electrodes 16 mounted on wearable body 12. Preferably, but not necessarily, system 10 comprises a plurality of sensing systems 40, each comprising several electrodes, so that electrodes 16 are physically grouped into a plurality of separate sensing systems 40. A more detailed description of sensing systems suitable for the present embodiments is provided hereinunder.

The number of electrodes 16 that are distributed over wearable body 12 is preferably at least 16 or at least 32 or at least 64 or at least 128 or at least 256 or at least 512 or more. Preferably, electrodes 16 are distributed over wearable body 12 at a density of at least 2 electrodes per 3 cm² or at least 4 electrodes per 3 cm² or at least 8 electrodes per 3 cm², or at least 2 electrodes per 2 cm² or at least 4 electrodes per 2 cm² or at least 8 electrodes per 2 cm², or at least 2 electrodes per cm² or at least 4 electrodes per cm² or at least 8 electrodes per cm². For at least 50% or at least 60% or at least 80% of the electrodes, the distance between adjacent electrodes is preferably less than 3 cm or less than 2.5 cm or less than 2 cm or less than 1.5 cm or less than 1 cm.

Use of large number of electrodes, according to preferred embodiments of the present invention allows system 10 to perform high-density EEG (hdEEG) which can provide high spatial sampling density as well as large head coverage.

hdEEG allows the recording of spontaneous or evoked brain activity with improved spatial resolution. hdEEG can provide spatial resolution (number of channels) that is sufficiently high for the investigation of various conditions such as, but not limited to, epilepsy, cognitive processes, brain Injury and neuropsychiatric disorders, as well as for detecting deep brain activities. hdEEG can be used, for example, for accurate localization of Epileptic foci, and/or neural network investigation, e.g., assessment of functional connectivity, and utilization in brain computer interface (BCI) systems.

A mounting configuration of electrodes 16 on wearable body 12 according to some embodiments of the present invention is illustrated in FIG. 1C. In these embodiments, electrodes 16 are mounted on flexible strings 32 that are attached gridwise to wearable body 12 (not shown in FIG. 1C, see, e.g., FIGS. 1A and 1B). In some embodiments, electrodes 16 are distributed uniformly along strings 32. In embodiments in which system 10 comprises a plurality of sensing systems 40, each comprising several electrodes, the sensing systems 40 are preferably, but not necessarily, distributed uniformly along strings 32.

In some embodiments of the present invention, an elastic band 34 is connected to strings 32. In the illustration shown in FIG. 1C, band 34 connects two strings 32 so that when band 34 is stretched (for example, while wearable body 12 is worn on scalp 14 strings 32 are displaced from each other along a direction generally orthogonal to strings 32, as generally shown by double-head arrow 36. Electrodes 16 can have any shape. Representative examples of shapes suitable for electrodes 16 on string 32 are illustrated in FIGS. 1D-F, showing a chain wheel shape (FIG. 1D), a tripod shape (FIG. 1E), and a disc or ball shape (FIG. 1F). Other shapes are also contemplated in some embodiments of the present invention.

Optionally, system 10 comprises a controller 72 having a circuit configured for controlling a connection state of each individual electrode. For example, controller 72 can group the electrodes electrically into two or more groups. The grouping is electrical in the sense that the signals sensed by all electrodes in a group are coherently combined so that the combined signal represents a sensing event from an area over the scalp 14 that is occupied by all the electrodes in the group. The groups can be defined based on one or more criteria. For example, in some embodiments of the present invention the groups are selected based on a predetermined morphology over a surface of the scalp, and in some embodiments of the present invention the groups are selected in a closed loop control based on an impedance or a signal-to-noise ratio of signals received by the electrodes, and in some embodiments of the present invention the groups are selected based on an independent signal analysis. Grouping operations according to some embodiments of the present invention are provided in greater detail hereinunder.

System 10 optionally and preferably also comprises a signal processor 18 having one or more circuits that receive and process EEG signals sensed by electrodes 16, and that optionally and preferably transmit control signals to controller 72. Processor 18 can be configured for executing any of the processing operations described herein. Typically, processor 18 comprises an Analog-to-Digital (A2D) circuit that digitizes the signals sensed by electrodes 16, and a digital signal processing (DSP) circuit that receives the digitized signals from the A2D circuit and applies digital signal processing operations to the digitized signals. Preferably, but not necessarily, processor 18 comprises a dedicated circuit, for example, an application-specific integrated circuit (ASIC) configured for executing these operations. Also contemplated is the use of a field-programmable gate array (FPGA) for performing at least a few of the image processing operations.

Communication between electrodes 16 and processor 18 is optionally and preferably by means of a wired bus interface, as further detailed hereinbelow. Processor 18 can be provided as a separate unit or be mounted on wearable body 12. Also contemplated are embodiments in which processor 18 comprises a first circuit that is mounted on wearable body 12 and a second circuit that is not mounted on wearable body 12, wherein one or more processing operations are performed by the circuit that is mounted on wearable body 12 and the other operations are performed by the circuit that is not mounted on wearable body 12. For example, the A2D circuit can be mounted on wearable body 12, and the DSP circuit can be distant to wearable body 12.

In some embodiments of the present invention processor 18 is configured for detecting Evoked Response Potential (ERP) signals from electrodes 16 and determining a physiological location of each electrode or each group of electrodes based on the ERP signals. In these embodiments, the density of the electrodes is preferably large (e.g., at least 2 electrodes per 3 cm² or at least 4 electrodes per 3 cm² or at least 8 electrodes per 3 cm², or at least 2 electrodes per 2 cm² or at least 4 electrodes per 2 cm² or at least 8 electrodes per 2 cm², or at least 2 electrodes per cm² or at least 4 electrodes per cm² or at least 8 electrodes per cm²), so as to improve the resolution of the location determination.

An ERP is the body's psychophysiological response to a given stimulus. Since individual neurons have relatively little electrical activity associated with them, certainly not enough to be detected on the scalp, ERPs are recorded when neurons act synchronously, and the electric fields generated by each particular neuron are oriented in such a way that the effects on the scalp cumulate. Activity of neurons organized in a layered open field manner (neurons with dendrites and axons oriented in the same fashion) are typically picked up as an ERP. Stimuli that cause ERPs can either be external, or internal.

The detection of ERP signals is optionally and preferably performed by processor 18 based on signals that describe stimulation of subject 22 wearing a wearable body 12. These signals are typically received from a stimulation system 20. Processor 18 preferably synchronizes between the signals from stimulation system 20 and the EEG signals from electrodes 16 to identify the ERP signals among the EEG signals. Stimulation system 20 can apply any type of stimulation, including, without limitation, somatosensory evoked potential (SSEP), brainstem evoked response audiometry, visual stimulation, tactile stimulation, olfactory stimulation and motor stimulation.

For any identified ERP signal, processor 18 can determine at which of electrodes 16 the ERP signal is sensed and determine the location of the electrode(s) based on the type of stimulus that evoked the ERP signal. In various exemplary embodiments of the invention several stimuli are applied to subject 22, and the identification of the respective ERP signal and the location of the electrode(s) that sense the respective ERP signal is repeated for each of at least a few of the applied stimuli. Preferably, a morphological map of the electrodes over the anatomical areas of the cortex is generated based on the determined locations. Thus, unlike conventional techniques, such as the 10-20 EEG scheme, in which each electrode must be placed at a specific location, the present embodiments advantageously generate a morphological map that automatically associates each electrode or group of electrodes to a specific anatomical area of the cortex.

For example, using SSEP sensory stimulation on the motor nerve system the maximal recorded P20 can localize the sensory cortex (post central gyrus). By localizing several anatomical locations and knowing the dispositions between the electrodes and sensing points, all electrodes can be mapped and tailored to individual's anatomy. The other electrodes can be mapped based on their physical distance from mapped electrodes. Another example is a stimulus in which visual patterns of specific images, such as, but not limited to, checkboards, or flicker lights, are displayed to induce visual evoked potentials can be induced. An additional example is a sound stimulus in which a specific sound (e.g., at a specific frequency, duration and/or amplitude) is presented to subject 22. Also contemplated, are electronic stimulations, such as, but not limited to, applying external stimulation to an organ, e.g., using a wrist stimulation device for applying an electric pulse to from the wrist. Such a stimulus induces an ERP signal in the sensory part of the brain and can thus be used to determine the location of the electrode(s) picking this signal. Further contemplated, are embodiments in which an electric pulse is transmitted to the scalp by one or more of the EEG electrodes and identifying the ERP signals indicted by this stimulation. In these embodiments, stimulation system 20 is optionally and preferably in direct communication with electrodes 16 and is configured to transmit electronic signals to the scalp via the respective electrode or electrodes.

Additional stimulation types and the corresponding cortex area at which an ERP signals is induced, are described in the Examples section that follows.

It is appreciated that multiplicity of stimulations can be applied simultaneously or sequentially. Preferably the applied stimulations are synchronous thereamongst.

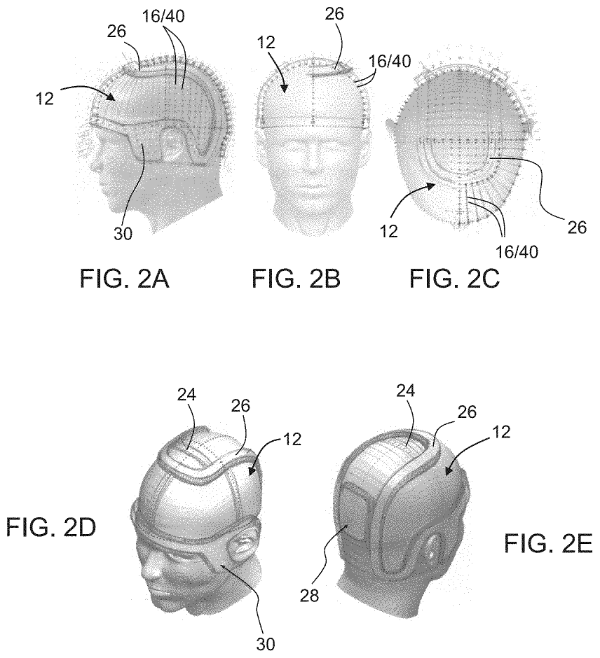

In some embodiments of the present invention wearable body 12 comprises a plurality of stretch sensors. These embodiments are illustrated in FIGS. 2A-E. Shown in FIGS. 2A-E are stretch sensors 24 mounted on wearable body 12. In some embodiments of the present invention stretch sensors 24 are woven into strings 32 (not shown, see FIG. 1C) on which electrodes 16 are mounted. Embodiments in which the stretch sensors are employed without strings 32 are also contemplated according to some embodiments of the present invention.

Wearable body 12 is preferably also provided with rigid arcs 26 for providing mechanical reinforcement to body 12 and may optionally include a hardware component 28 that may include one or more a bus architecture and a controller, as further detailed hereinbelow, and/or a power source 30. Additional configurations for wearable body 12 are described hereinunder.

Stretch sensors 24 convert analog motion or tension to stretch data describing a stretching of wearable body 12. Signal processor 18 (not shown, see FIGS. 1A and 1B) optionally and preferably receives the stretch data from sensors 24 and constructs from the stretch data a three-dimensional map describing the locations of electrodes 16 over the scalp, once wearable body 12 is stretched. Sensors suitable for use as stretch sensors 24 are disclosed in U.S. Published Application No. 20160238368, and are commercially available from StretchSense™, Auckland, New Zealand.

Typically, processor 18 constructs the three-dimensional map also using one or more reference points for which the locations are known. In these embodiments the wearable body is worn and stretched such that one or more reference points that are marked on the wearable body are aligned with specific anatomical points (e.g., ears, eyebrows, forehead midline, etc.). Preferably, signal processor is programed in advance with information pertaining to the length in rest state of the elastic band 34, or the inter-string distance when elastic band 34, is not stretched. Using this information and the stretch data from sensors 24, processor 18 calculates the inter-electrode distances, and using the calculated distances and the location of the reference points, processor 18 constructs the three-dimensional map.

The three-dimensional map allows system 10 to create a cross-correlation between the brain activity axis system and the physical positioning of electrodes 16 and localize a specific area of interest at both the brain simulation and the scalp on the anatomical level with high precision.

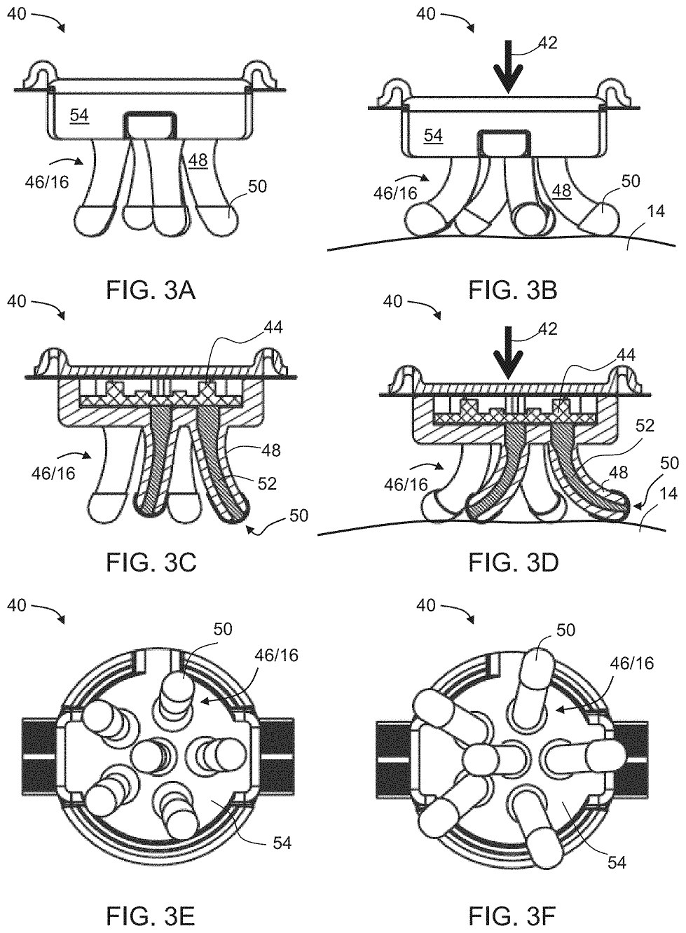

Reference is now made to FIGS. 3A-F, 4A-F, and 5A-F, which are schematic illustrations of a sensing system 40 that comprises several electrodes 16, according to various exemplary embodiments of the present invention. Shown are front views (FIGS. 3A-B, 4A-B, and 5A-B), cross-sectional views (FIGS. 3C-D, 4C-D, and 5C-D), and bottom view (FIGS. 3E-F, 4E-F, and 5E-F) of system 40 in a relaxed state (FIGS. 3A, 3C, 3E, 4A, 4C, 4E, 5A, 5C, and 5E) and in a compressed state (FIGS. 3B, 3D, 3F, 4B, 4D, 4F, 5B, 5D, and 5F), once a compression force 42 is inwardly applied to sensing system 40 in the direction of scalp 14. A plurality of sensing systems like system 40 can be distributed over wearable body 12 of system 10, for example, along strings 32, as further detailed hereinabove.

Sensing system 40 typically comprises a circuit board 44, e.g., a printed circuit board, and a plurality of flexible sensing legs 46. Optionally, each of legs 46 has a non-conductive section 48 and a tip 50 in electrical communication with circuit board 44 via a conductive section 52. In some embodiments of the present invention sections 48 and 52 are aligned in a core-shell relationship wherein non-conductive section 48 at least partially surrounds conductive section 52, but this need not necessarily be the case, since in some embodiments of the present invention sections 48 and 52 are aligned in other configurations, such as, but not limited to, a side-by-side configuration.

In some embodiments of the present invention one or more of legs 46 does not include separate non-conductive and conductive sections. For example, one or more of legs 46 can have an interlaced structure in a manner that non-conductive regions are interlaced with conductive regions. For example, the leg can be made of a non-conductive material, such as, but not limited to, a non-conductive polymeric material, impregnate with conductive particles, such as, but not limited to, carbon particles or metallic (e.g., nickel) particles.

Preferably, circuit board 44 is detachable from legs 46. These embodiments are advantageous since they allow making system 46 partially disposable and partially reusable. Specifically, legs 46, which are in contact with the scalp, can be made disposable, and circuit board 44 can be made reusable. Prior to the mounting of system 10 onto the head of the subject, new disposable legs 46 are attached to circuit board 44 so that it is not necessary to use the same legs for different subjects or different sessions.

Circuit board 44 serves for collecting signals for each individual section 52 and transmitting it to processor 18, optionally and preferably via a wired bus interface, as further detailed hereinbelow.

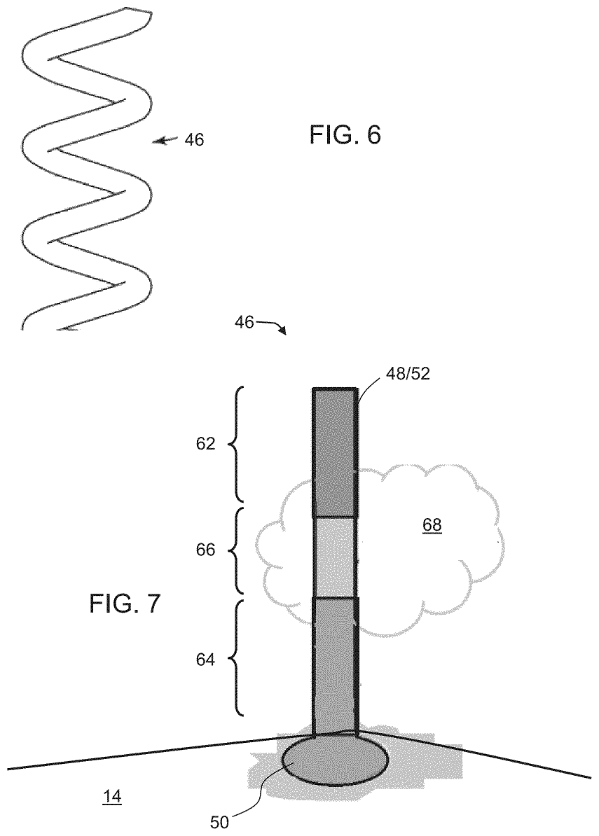

Each leg 46 of system 40 thus enacts one of electrodes 16 of system 10. Legs 46 can be of any three-dimensional shape that facilitates its bending or collapse towards scalp 14 once wearable body 12 is worn thereon, and once force 42 is applied. FIGS. 3A-5E illustrate a curved shape for legs 46, wherein the compression force 42 increases the curvature of the shape. Also contemplated are embodiments in which one or more of legs 46, more preferably each of legs 46, has a helical shape. A schematic illustration of a leg 46 in these embodiments is illustrated in FIG. 6. In various exemplary embodiments of the invention the shape and orientation of legs 46 is selected that at least one, more preferably each, of leg 46 is oriented along a direction that is non-coincidental with the normal direction to the scalp 14.

In some embodiments, circuit board 44 is configured for pre-processing the signals sensed by legs 46. For example, circuit board 44 can include a buffering amplifier and/or be configured for providing active shielding to reduce noise.

It was found by the Inventors that sensing system 40 is capable of penetrating through the hair of subject 22 and also to provide improved contact with scalp 14, optionally and preferably in dry environment.

Preferably, legs 46 are made, at least in part, of a shape memory material, such as, but not limited to, a shape memory alloy. The shape memory material can be incorporated in the flexible section 48, in which case the shape memory material is preferably a non-conductive polymer or a conductive material (e.g., a conductive polymer or alloy) that is coated by a non-conductive coating to insulate section 52 from other conductive sections. Alternatively or additionally, the shape memory material can be incorporated in the section 52, in which case the shape memory material is preferably a conductive polymer or alloy.

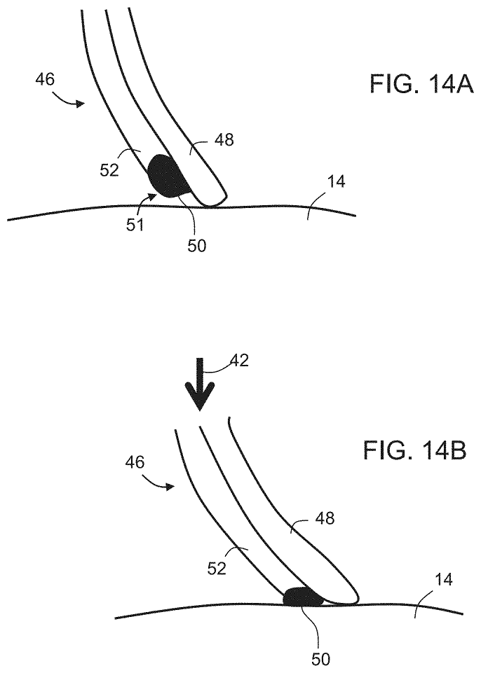

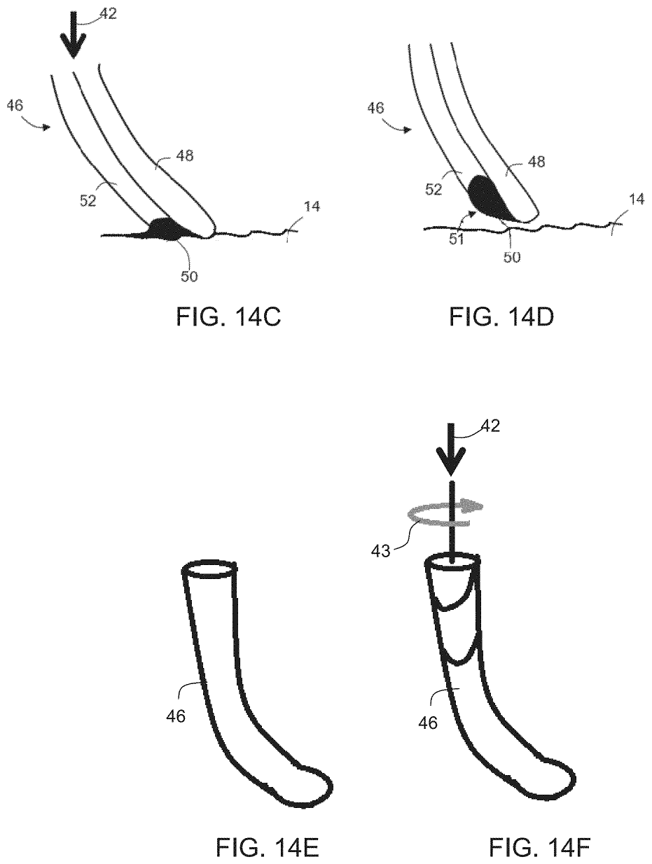

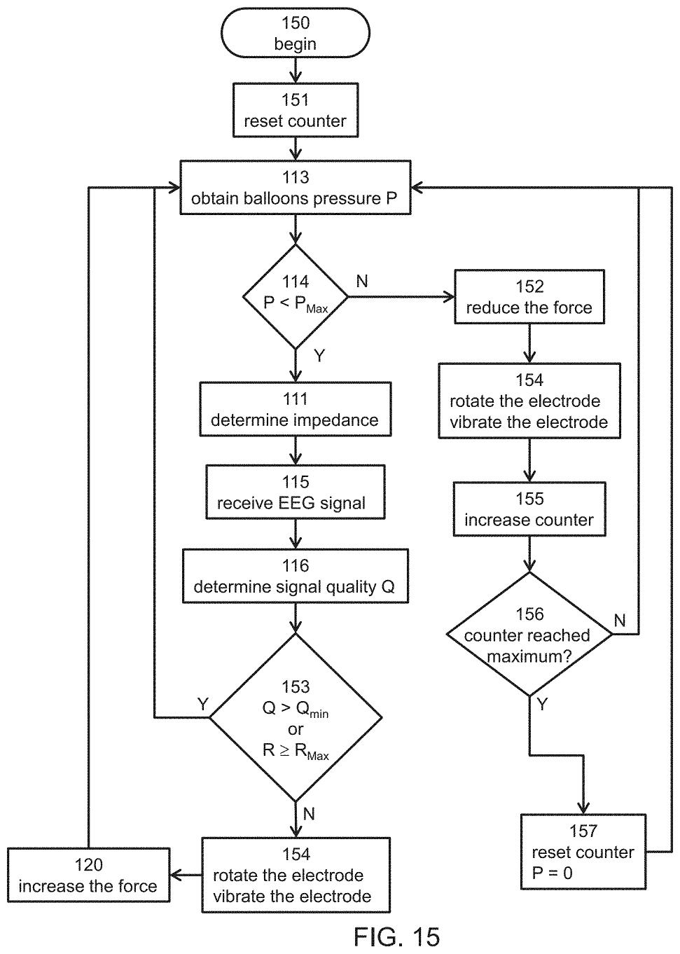

In some embodiments of the present invention electrical conductance between scalp 14 and circuit board 44 is established only after force 42 is applied, wherein before the application of force 42 scalp 14 and circuit board 44 are devoid of electrical conductance therebetween. These embodiments are advantageous since they prevent short circuit in case tips of neighboring sensing systems overlap. These embodiments can be realized by ensuring that tip 50 of leg 46 contacts scalp 14 only after force 42 is applied. A preferred configuration for this realization is illustrated in FIGS. 14A and 14B. Shown is a leg 46 in which the conductive 52 and non-conductive 48 sections are aligned in a side-by side configuration. In the absence of force 42 (FIG. 14A), leg 46 is oriented and assumes a shape in which non-conductive 48 section contacts scalp 14 and maintains a gap 51 between tip 50 and scalp 14. Following the application of force 42 (FIG. 14B), leg 46 experiences a strain and assumes a shape in which the end of conductive section 52 comes in contact with scalp 14.

In some embodiments of the present invention tip 50 is constructed and designed such that its contact area with scalp 14 is increased after the application of force 42, as illustrated in FIGS. 14C and 14D. The cross-sectional area of tip 50 in a plane generally parallel to scalp 14 is smaller in the absence of force 42 (FIG. 14D) than in the presence of force 42 (FIG. 14C). The advantage of such a configuration is that it improves the electrode tissue impedance. Preferably, the material of tip 50 is sufficiently soft so that once it is strained by force 42 its surface conforms with the shape of the skin in contact therewith and fills or partially files skin grooves (FIG. 14C).

In some embodiments of the present invention legs 46 are arranged such that upon application of compression force 42, the legs rotate towards the scalp 14. These embodiments are illustrated in FIGS. 14E and 14F, showing leg 46 before (FIG. 14E) and after (FIG. 14F) the application of force 42, wherein leg 46 is arranged to rotate 43 in response to force 42. The rotation can be effected by means of the arrangement of legs 46 over a base of a body 54 of system 40 (not shown in FIGS. 14A-D, see FIGS. 3A-5F). For example, the legs can be arranged in a spiral manner so that once force 42 is applied, the spiral arrangement ensures transforming the inwardly applied force into a turning force. Alternatively or more preferably additionally, force 42 is applied while establishing rotary motion to legs 46. The conductive 52 and non-conductive 48 sections of leg 46 are not shown in FIGS. 14E and 14F, but the ordinarily skilled person, provided with the information described herein, would know how to construct leg 46 to include the conductive 52 and non-conductive 48 sections.

Combination of the embodiments illustrated in FIGS. 14A-B and/or FIGS. 14C-D with the embodiments illustrated in FIGS. 14E-F are also contemplated. In this combination of embodiments, force 42 both causes leg 46 to rotate and applies a strain on leg 46 so that tip 50 comes in contact with scalp 14.

Also contemplated are embodiments in which a separate force is applied for rotating the leg.

Preferably, the tip 50 is made of soft material with low shore (e.g., Shore OO Hardness of from about 10 to about 100), that experiences a strain once force 42 is applied, as illustrated in FIGS. 14B and 14C. These embodiments are useful from the standpoint of user's comfort and are also advantageous since the strain increases the footprint on scalp 14.

Shore OO Hardness describes a material's resistance to permanent indentation, defined by type OO durometer scale. Shore A hardness is typically determined according to ASTM D2240-00.

A representative example of a material from which tip 50 can be made include, without limitation, silicon coated with AgC1, or Polyurethane composites with metal, or Carbon Black, but other conductive materials are also contemplated.

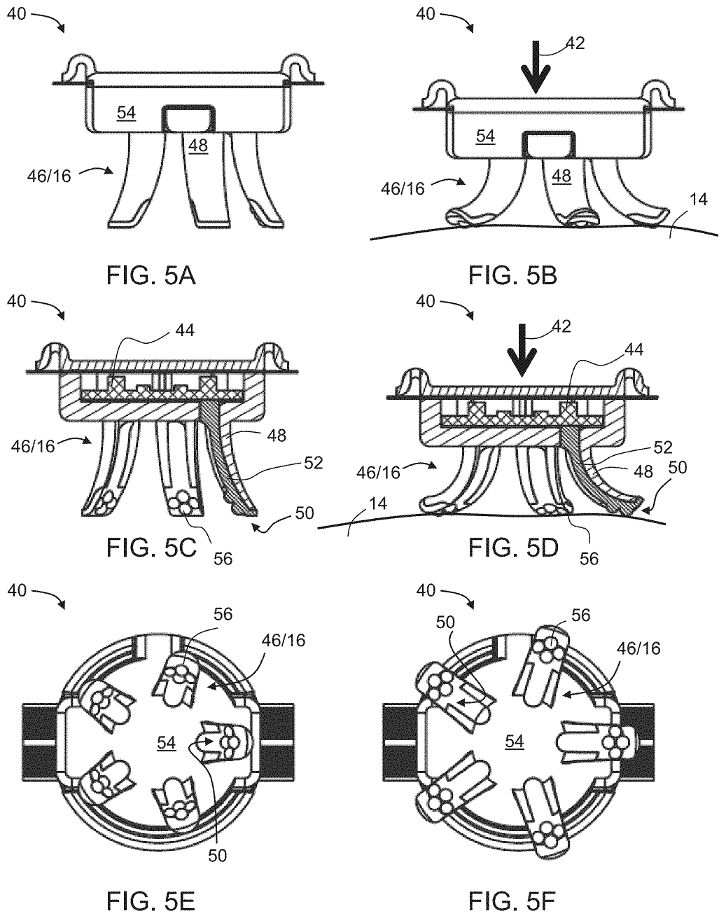

In some embodiments of the present invention tip 50 is constructed and designed such that a friction force between tip 50 and scalp 14 is sufficient to prevent movement of tip 50 from its location after the application of force 42. Typically, but not necessarily, tip 50 is coated with a coating having a plurality of micrometric or nanometric structures 56 (FIGS. 5C-F), so as to increase the friction force and also increase the surface area of tip 50 hence to improve the electrical conductivity of tip 50.

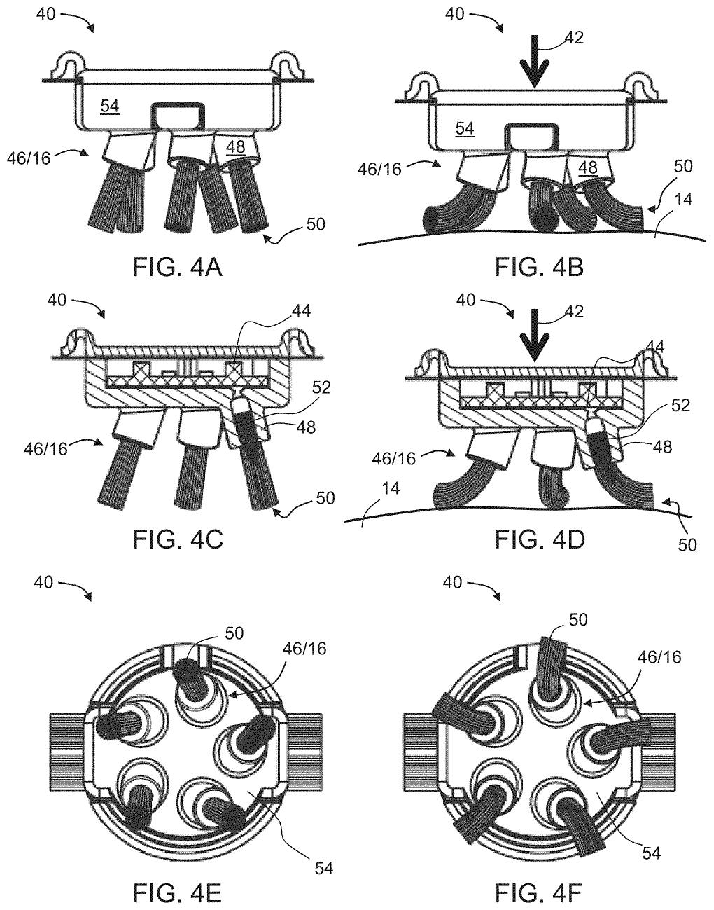

The present embodiments contemplate several types of conductive sections for legs 46. In some embodiments of the present invention the conductive section 52 of one or more of legs 46 is a metallic wire. These embodiments are illustrated in FIGS. 3A-F. In some embodiments of the present invention the conductive section 52 of one or more of legs 46 comprises a bundle of conductive bristles. These embodiments are illustrated in FIGS. 4A-F. In some embodiments of the present invention the conductive section 52 of one or more of legs 46 is made of a conductive polymer. These embodiments are illustrated in FIGS. 5A-F. Combination of these embodiments, e.g., a sensing system with at least two legs 46 with different types of sections, are also contemplated.

Referring in particular to FIGS. 4A-F, each bristle of section 52 optionally and preferably have a thickness of from about 0.05 mm to about 0.4 mm, e.g., 0.15 mm. The advantage of having bundle of conductive bristles for section 52 is that it facilitates penetration into the hair of the subject. Another advantage is that the tip 50 of the bundles conforms with the shape of scalp 14, whereby different bristles of the bundle may engage points scalp 14 that have different heights resulting in enlarged footprint of system 40 on the scalp. In some embodiments of the present invention bundles are arranged in a spiral manner (FIGS. 4E and 4F) over a base of a body 54 of system 40, so as to prevent bristles of one bundle to contact with bristles of another bundle. Once force 42 is applied, the spiral arrangement ensures transforming the inwardly applied force into a turning force that acts as a screw on the tip of the bristles. The advantage of using the bristles is that it reduces the likelihood that the electrodes will be entangled with the subject's hair.

Reference is now made to FIG. 7 which is a schematic illustration of a leg 46 of system 40, in embodiments in which the non-conductive section 48 and/or the conductive section 52 of leg 46 comprises a hydrophobic zone 62 and a hydrophilic zone 64.

Preferably, hydrophobic zone 62 is at the upper part of leg 46 (not in contact with the scalp) and hydrophilic zone 64 is at the lower part of leg 46 (adjacent to tip 50). The advantage of this embodiment is that humidity, resulting, e.g., from sweat at the surface of scalp 14, is concentrated at the vicinity of the lower part of leg 46 increasing the conductivity with the human tissue, and prevents it from arriving at the circuit board 44 (not shown). This embodiment is also advantageous since it prevents electricity shorts between adjacent sensor systems.

In some embodiments of the present invention leg 46 also comprises an intermediate zone 66 between hydrophobic zone 62 and hydrophilic zone 64. Intermediate zone 66 is less hydrophobic than hydrophobic zone 62, and less hydrophilic than hydrophilic zone 64. For example, intermediate zone 66 can be neither hydrophilic nor hydrophobic. In some embodiments the hydrophobicity of intermediate zone 66 is gradually increased in the upward direction. The advantage of having intermediate zone 66 is that the temperature differences between scalp 14 and wearable body 12 create a sink at the vicinity of zone 64. Since the temperature is typically higher at scalp 14 than at wearable body 12, air vapors 68 tends to condense at the higher parts of leg 46. Since the upper part is hydrophobic, the vapor 68 condenses at a region on the intermediate zone 66 and be drawn downwards by the hydrophilic zone 64 acting as a vapor sink. This prevents electrical shorting between adjacent legs of the same sensing system and/or between adjacent legs of adjacent sensing systems.

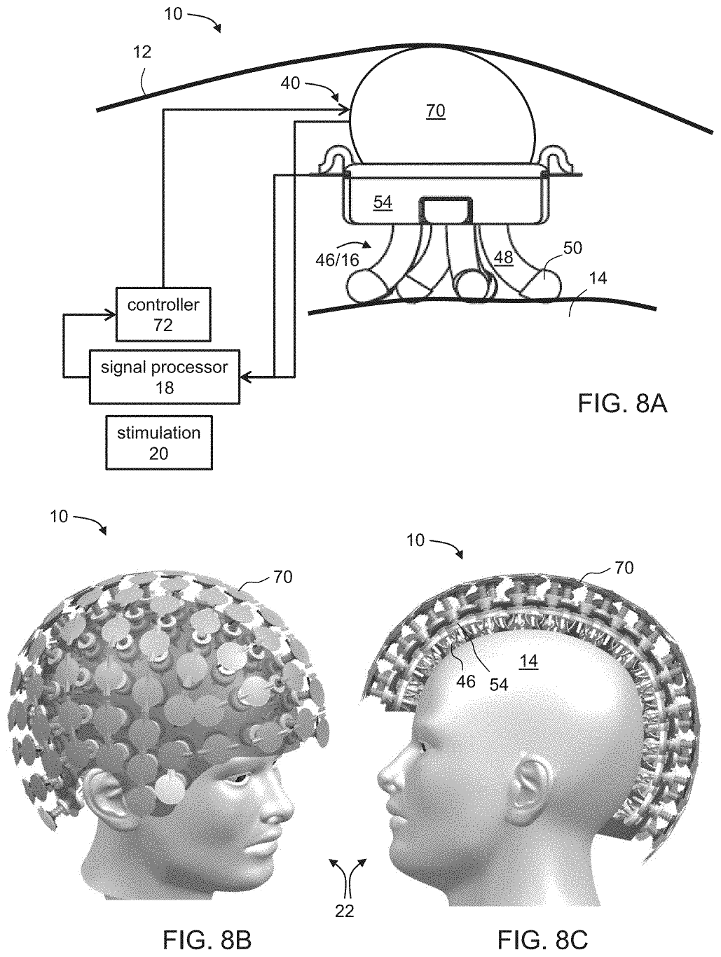

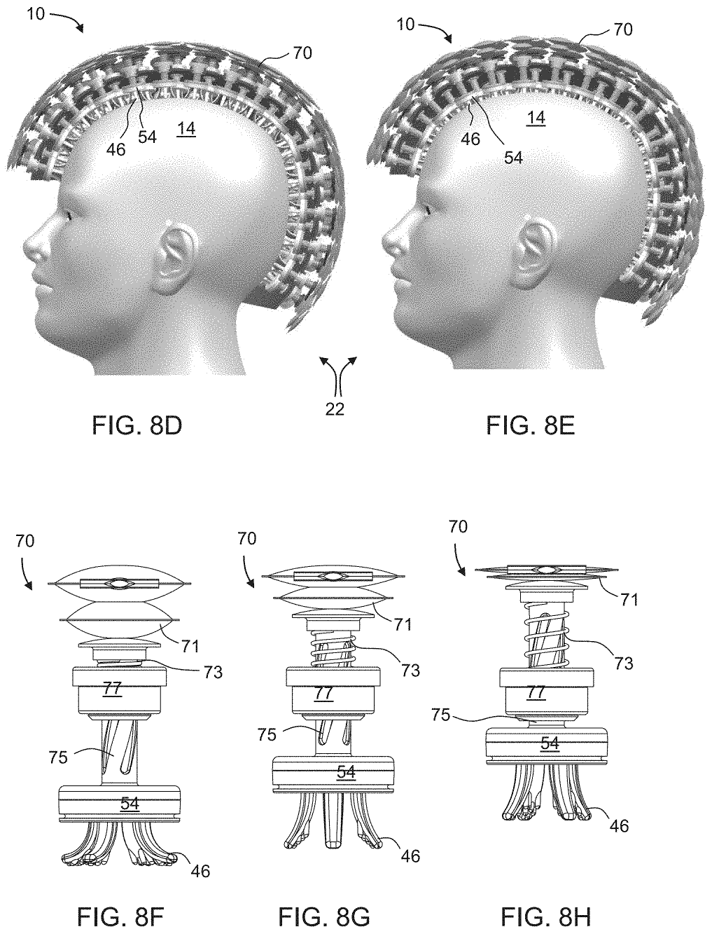

FIGS. 8A-H are schematic illustration of system 10 in embodiments of the invention in which system 10 comprises controllable actuators for applying force to the electrodes. In the illustrations shown in FIGS. 8A-H, which is not to be considered as limiting, the electrodes of system 10 are grouped into sensing systems 40, but this need not necessarily be the case, since configurations in which the electrodes are not grouped are also contemplated, according to some embodiments of the present invention. For clarity of presentation, only one sensing system 40 is shown in FIG. 8A, but it is to be understood that many sensing systems like system 40 can be incorporated in EEG system 10.

In the illustrated embodiment, system 10 comprises a plurality of controllable actuators 70, for applying force to electrodes 16 or legs 46. For clarity of presentation, one actuator is magnified in FIG. 8A. Actuators 70 are designed and configured to apply to electrodes 16 or legs 46 a compression force (e.g., force 42), optionally and preferably a non-periodic compression force. Actuators 70 can in some embodiments of the present invention be designed and configured to apply to electrodes 16 or legs 46 a periodic (e.g., sinusoidal) force so as to vibrate the legs or electrodes or to generate a hammering effect, facilitating better penetration of the electrodes or legs through the and better spread of the contact area between the tips and the scalp. A hammering effect can be generated by controlling actuator 70 to periodically apply and terminate the application of a compression force to the electrodes. Actuators 70 can in some embodiments of the present invention be designed and configured to apply to electrodes 16 or legs 46 a rotating force so as to rotate the legs, for example, about a longitudinal axis of the legs, as illustrated in FIG. 14F.

Preferably, but not necessarily, each actuator 70 is mounted on top of the body 54 of sensing system 40.

Further contemplated, are combinations of the above embodiments. For example, one or more actuators can apply a compression force (e.g., force 42), and one or more actuators can apply a periodic force to vibrate the legs or to generate a hammering effect.

FIGS. 8B-E schematically illustrate system 10 once mounted on scalp 14 of a subject 22, FIG. 8B is a perspectives view, and FIGS. 8C, 8D and 8E are cross-sectional views, at three different states of actuators 70. Specifically, in FIG. 8C actuators 70 are in a relaxed state in which no force is applied to legs 46, in FIGS. 8D and 8E actuators 70 are apply a force to legs 46, wherein the state shown in FIG. 8E correspond to a stronger force compared to the state shown in FIG. 8D, thereby bringing body 54 closer to scalp 14 and applying more strain to legs 46 creating higher footprint areas between the tips and the scalp.

Each of actuators 70 can be embodied, for example, as inflatable balloon, applying force upon inflation thereof, a piezoelectric actuator or an electric actuator (e.g., a solenoid) or motor applying force upon application of voltage thereto, a pneumatic actuator applying force by means of a motion of a piston, etc.

FIGS. 8F-H schematically illustrate actuator 70 in embodiments in which actuator 70 comprises an inflatable balloon 71. In these embodiments, actuator 70 can also comprises a retraction mechanism 73, which can be embodied as an elastic member, e.g., a spring, a rotating shaft 75 and an annular stator 77. Shaft 75 is positioned within stator 77. The proximal end of shaft 75 engages balloon 71 and the distal end of shaft 75 is connected to body 54. Preferably, shaft 75 is provided with one or more helical grooves and the inner surface of stator 77 is provided with one or more protrusions fitting into the grooves of stator 77, so that stator 77 and shaft 75 serve as a push-to-rotate mechanism. Upon inflation of balloon 71, shaft 75 is pushed through stator 77 in the direction of the scalp (not shown in FIGS. 8F-H) and is being rotated by stator 77. Body 54, which is connected to the distal end of shaft 75, rotates while applying the force on legs 46. Once balloon 71 is deflated, retraction mechanism 73 retracts shaft 75 through stator 77.

FIGS. 8F-H illustrate three states of actuator 70. FIG. 8F illustrates a state at which the force applied to legs 46 is sufficiently high to strain the legs, FIG. 8G illustrates a state at which the force applied to shaft 75 is sufficient to establish contact between legs 46 and the scalp, and FIG. 8H illustrates a state at which shaft 75 is fully retracted by mechanism 73, until legs 46 do not contact the scalp. A typical inflation pressure P_{c} to establish contact between legs 46 and the scalp (FIG. 8G) is a differential pressure (relative to the ambient pressure) of from about 2 to about 12, e.g., about 4 PSI. When the differential pressure within balloon 71 is above P_{c} legs 46 are pressed against the scalp and experience strain (FIG. 8F), and when the differential pressure within balloon 71 is less than P_{c} shaft 75 is retracted by mechanism 73 (FIG. 8H). Other pressure levels are also contemplated. In various exemplary embodiments of the invention the pressure within balloon 71 at the state at which legs 46 are pressed against the scalp is selected such that the load of legs 46 on the scalp is not more than an empirical parameter (e.g. 3 N/cm²).

When actuator 70 of system 40 comprises balloon 71, shaft 75 and stator 77, wearable body 12 optionally and preferably comprises an outer shell 12a and an inner shell 12b. FIGS. 9A and 9B showing an exploded view (FIG. 9A), an assembled view (FIG. 9B) of system 10. In the illustrated embodiments, outer shell 12a serves for supporting balloon 71 and pneumatic tubes 162 for inflating and deflating balloon 71, and inner shell 12b serves for supporting other components of sensing system 40, including at least stator 77, circuit board 44, and body 54 with legs 46. Optionally and preferably, at least one of shells 12a and 12b, more preferably both shells 12a and 12b, is rigid. Preferably, the size of inner shell 12b is adjustable to fit onto scalp 14.

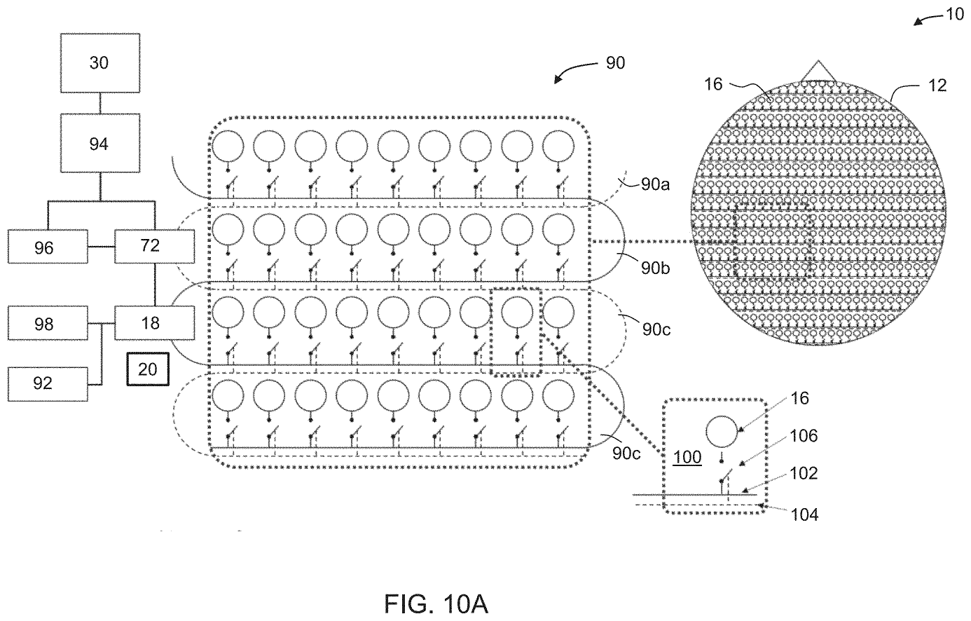

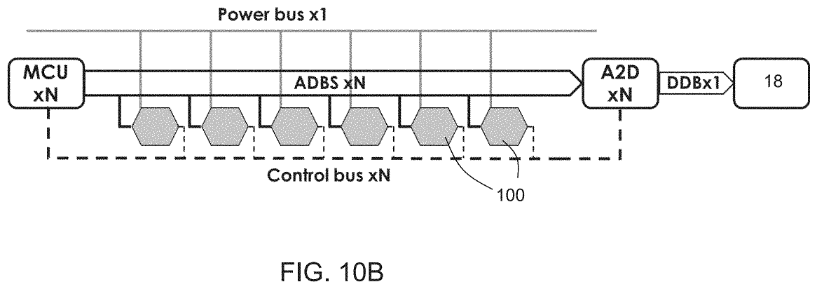

Typically, system 10 also comprises a wired bus interface 90 for establishing electrical contacts among various components of system 10. Interface 90 can be mounted on inner shell 12b. A more detailed description of a bus interface suitable for the present embodiments is provided hereinunder with reference to FIGS. 10A and 10B.

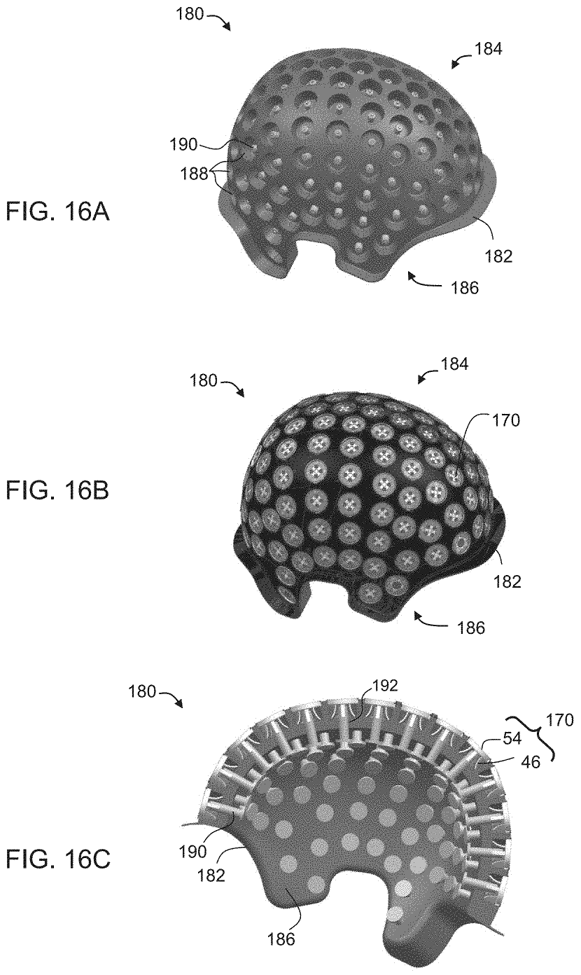

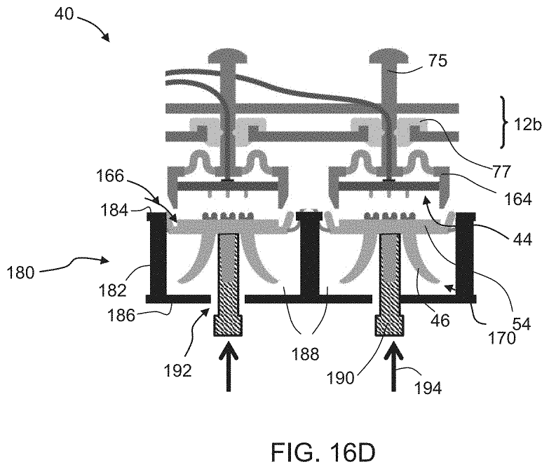

Circuit board 44 is preferably detachable from body 54, and can be enclosed in a housing 164, designed for receiving body 54. In some embodiments of the present invention housing 164 comprises a rigid wall 165 and a flexible membrane 167 connecting wall 165 to shaft 75. The advantage of these embodiments is that they provide flexibility in the orientation of housing 164 with respect to shaft 75, hence to allow the sensing systems 40 to conform with the shape of the scalp. Male and female mating snap members 166 are optionally and preferably employed for facilitating fast connection and detachment between circuit board 44 and body 54. A preferred technique for connecting body 54 to housing 164 is by means of a jig system, as described hereinunder with reference to FIGS. 16A and 16B.

In some embodiments of the present invention bodies 54 of two or more of, optionally and preferably all of, sensing systems 40 are interconnected by means of a sheet member 168, which is optionally and preferably shaped as a cap. Typically, bodies 54 are embedded within sheet member 168. A perspective view of sheet member 168 (shaped as a cap), with interconnecting sensing systems 40 embedded therein is illustrated in FIG. 9C. Sheet member 168 is preferably made flexible to provide each sensing system 40 with independent rotational and translational motion. In some embodiments of the present invention bodies 54 are securely connected to member 168 to form a unitary structure that cannot be disassembled by the end user without causing a physical damage (e.g., rupture, breakage). Optionally and preferably, bodies 54 and member 168 form a disposable and non-disassemblable structure.

FIG. 9B illustrates an exemplified situation in which the balloon 71 of one sensing system is inflated to ensure contact between the legs 46 and the scalp 14, and the balloon 71 of an adjacent sensing system is deflated so that there is no contact between the legs 46 and the scalp 14. Body 54 with legs 46 and optionally also flexible member 168 are collectively referred to herein as electrode assembly 170.

Referring again to FIG. 8A, system 10 optionally and preferably comprises a controller 72 configured for individually controlling each actuator 70 to apply the force. Controller 72 receives control signals from signal processor 18. Signal processor 18 can signal controller 72 to operate actuator 70 following a user input, for example, when the subject 22 feels discomfort, the subject 22 can enter a command to processor 18 to release the pressure on the electrodes or to initiate a vibration protocol. Alternatively or additionally, signal processor 18 and controller 72 operate in a closed loop control, wherein signal processor 18 receives and processes EEG signals from the electrodes (e.g., legs 46 or electrodes 16 of the sensing systems 40) and transmits the control signals to controller 72 based on the processing.

Typically, signal processor 18 determines electrode-tissue impedance or one or more signal quality measures and transmits the control signals to controller 72 based on the determined impedance.

As used herein “electrode-tissue impedance” (ETI) is a ratio of the voltage drop between two given electrodes to the current flowing between those two electrodes.

For example, signal processor 18 can signal controller 72 to increase a compressive force applied by actuator 70 when the determined impedance is above a predetermined threshold, and release it otherwise. This allows system 10 to operate at low impedance values at all times. Controller 72 can receive control signals for controlling actuator 70 also when actuator 70 vibrates the electrodes or generates a hammering effect. Typically, signal processor 18 signals controller 72 to initiate a vibration or hammering protocol by actuator 70 when the determined impedance is above a predetermined threshold.

The advantage of executing vibration and/or hammering protocol is that it allows to relocate the sensing system and to improve comfort to the user.

Signal processor 18 can also receive from actuator 70 signals indicative the force or pressure it applies to the electrodes or legs. For example, when actuator 70 comprises an inflatable balloon, signal processor 18 can receive from actuator 70 signals describing the pressure within the balloon. The signals that are indicative of the force or pressure can be used by signal processor 18 in selecting the control signals it transmits to controller 72. In an embodiment of the invention, signal processor 18 signals controller 72 to increase a compressive force applied by actuator 70, in response to a determination that (i) the determined impedance is above the predetermined, and (ii) that the applied force is below a predetermined threshold; and to initiate a vibration or hammering protocol by activation actuator 70 to apply a periodic force, in response to a determination that (i) the determined impedance is above the predetermined, and (ii) that the applied force is equal to or above the predetermined threshold. A detailed procedure suitable for implementing a closed loop control is provided below.

Reference is now made to FIG. 10A which is a schematic illustration of system 10 in embodiments of the invention in which signal transmission among various components of system 10 is via bus architecture. Sown in FIG. 10A are wearable body 12, electrodes 16, processor 18, stimulation system 20, and controller 72 as further detailed hereinabove. Further illustrated is a wired bus interface 90. Preferably, bus interface 90 comprises a plurality of bus rows 90a, 90b, etc., each distributing signals from and to several electrodes on wearable body 12 (e.g., a row of electrodes). Also illustrated is power source 30 and a power block 94 that distributes power to the other components of system 10, a human machine interface 96 for allowing the operator to enter commands, select parameters, or otherwise reconfigure controller 72 and/or processor 18, a wire data interface 98 via which communication is established between processor 18 and bus interface 90. A communication system 92 employing a communication technique, such as, but not limited to, Wi-Fi, Bluetooth®, wireless telephony or the like may also be included in system 10 for allowing system 10 to communicate, via wire data interface 98, with an external system, such as a computer, a cloud computing facility, a cloud storage facility or the like.

For each of the rows of bus interface 90, all the corresponding electrodes share same galvanic bus wire, and the EEG data is gated by controller 72. With specific reference to FIG. 10A, each of the rows of bus interface 90 comprises a data bus 102, a control bus 104, and a plurality of bus cells 100. The data bus 102 and control bus 104 are shared by all the electrodes corresponding to the respective bus row. The data bus 102 can be an analog or digital data bus, depending whether digitization is executed before or after transmission of the EEG signals to processor 18. Each cell 100 is addressable by controller 72 and comprises a control gate 106 that individually controls the connection state of a specific electrode 16. Optionally and preferably each electrode publishes over the bus row a digital identification for allowing controller 72 to identify the bus cell that controls the respective electrode. Controller 72 specifically addresses the respective bus cell 100 and transmits gating signals to switch the respective electrode between a connected and unconnected state.

When it is desired to collect EEG signals globally from a region-of-interest over the scalp, controller 72 electrically groups the electrodes that morphologically cover or able to sense signals from the region-of-interest, by transmitting gating signals to the respective cells, wherein the desired electrodes are switched, in a parallel manner, to a connected state. The signals from the electrodes that morphologically cover or able to sense signals from the region-of-interest are combined, optionally and preferably coherently, thereby allowing magnification of the signal collected from the region-of-interest. Controller 72 can electrically group electrodes also when processor 18 determines, based on signal processing analysis, that the quality of the signal is reduced, for example, based on the electrode-tissue impedance or signal-to-noise ratio. For example, when processor 18 determines that the impedance at electrodes that are within a specific area over the scalp is above a predetermined threshold, processor 18 can signal controller 72 to electrically group those electrodes. When processor 18 determines that the impedances at electrodes that cover several distinct areas over the scalp are above a predetermined threshold, processor 18 can signal controller 72 to electrically group each of those electrodes to one or more neighboring electrodes.

FIG. 10B is a schematic illustration of the architecture of a single (out of N) row bus, according to some embodiments of the present invention. A power bus provides power to all the bus cells 100. The power bus can be shared by all the row buses. A main control unit MCU, which is a part of controller 72 (FIG. 10A) that is allocated to the specific row bus, optionally issues via the control bus commands for scanning, grouping, sampling and data retrieval based on the electrode's digital identification. The data bus optionally and preferably comprises an analog data bus (ADBS), which can in some embodiments of the present invention gather the raw analog EEG signals to an analog to digital (A2D) converter. The raw sampled signal may be of single electrodes or a group of electrodes, if electrode grouping is employed. The main control unit can use the control bus to synchronize the sampling and direct the sampled signals from the electrodes to the A2D converter. From the A2D converters a digital data bus (DDB) optionally and preferably gathers all EEG data to processor 18.

One advantage of bus interface 90 (FIG. 10A) is that it reduces the need for individual cables that would otherwise be required for each electrode, thereby reducing the weight and complexity of the system. Another advantage of bus interface 90 is that the mapped location of the electrodes allows simple routing of orders and data retrieval to and from electrodes according to specific neurological event in the brain. In various exemplary embodiments of the invention system 10 employs high rate electronics (e.g., sampling rate of at least 200 Hz or at least 300 Hz or at least 400 Hz or at least 500 Hz, with word length of at least 32-bit or at least 64-bit). This allows system 10 to switch and sample each electrode at the required EEG frequency band, while digitally sampling EEG signals by grouping electrodes per dedicated assignments. Another advantage of bus interface 90 is that it allows multi drop reading of the electrode, wherein readouts can be collected serially using the same bus channel. Another advantage of bus interface 90 is that the power management is improved, since it is not necessary to activate all electrodes at the same time.

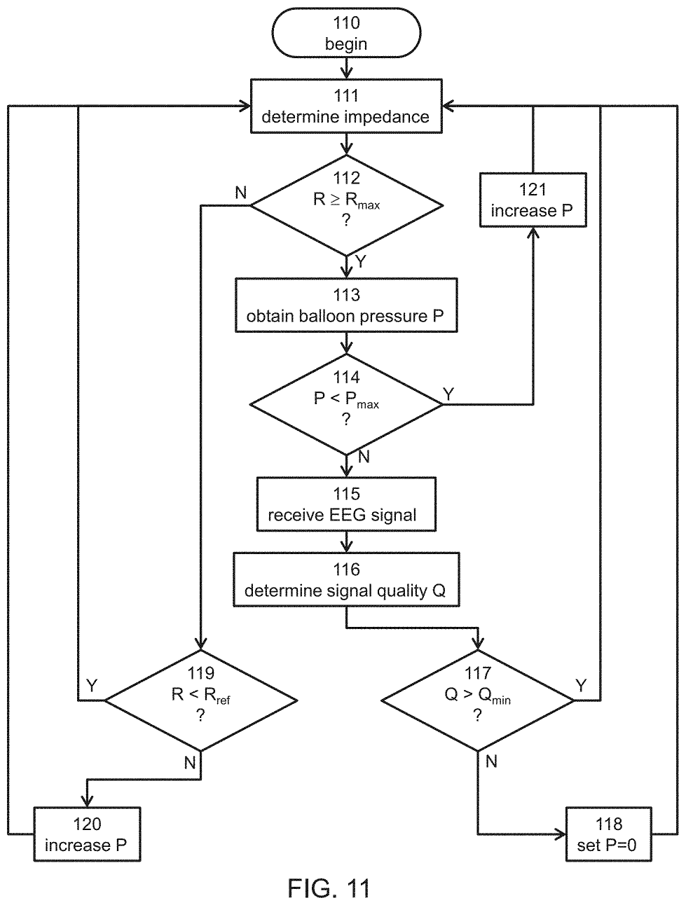

FIG. 11 is a flowchart diagram describing a close loop procedure for operating an actuator to apply a compression force on an electrode, according to some embodiments of the present invention. The procedure can be executed, for example, by system 10, particularly by actuator 70, electrodes 16, processor 18 and controller 72. The procedure begins at 110 and continues to 111 at which the electrode-tissue impedance is determined, for example, by signal processor 18. The procedure continues to decision 112 at which the impedance is compared to a first predetermined threshold Rₘₐₓ.

A representative example of a value for the first predetermined threshold Rₘₐₓ is from about 50 KΩ to about 250 KΩ.

If the impedance is less than Rₘₐₓ, the procedure continues to decision 119 at which the impedance is compared to a second predetermined threshold R_{ref}.

A representative example of a value for the second predetermined threshold R_{ref} is from about 60 KΩ to about 180 KΩ.

If the impedance is above or equal to R_{ref} the procedure proceeds to 120 at which a compression force applied to the electrode is increased. From 120 the procedure loops back to 111.

If, at decision 112, the impedance is above or equal to Rₘₐₓ, the procedure continues to 113 at which a value of a parameter indicative of the force applied by the actuator is obtained. For example, when the actuator comprises a balloon, the procedure can obtain the pressure within the balloon. The procedure continues to decision 114 at which the parameter (pressure, in the present example, preset as comfortability of user threshold) is compared to a predetermined threshold Pₘₐₓ indicative of an upper limit of the force that is to be applied (upper limit of a pressure, in the present example).

A representative example of a value for the predetermined parameter threshold Pₘₐₓ, is from about 25 cmH₂O to about 500 cmH₂O, e.g., about 250 cmH₂O.

If the parameter is below the Pₘₐₓ, the procedure continues to 115 at which an EEG signal is received from the electrodes and to 116 at which the quality of the signal is determined. This is optionally and preferably done by processor 18, and may include calculating at least one of the impedance, the power and the frequencies in the signal.

The procedure continues to decision 117 at which the quality of the signal is compared to a quality threshold or a set of quality thresholds. For example, data processor 18 can calculate a score Q using the calculated quantities and compares the score to a predetermined score threshold Qₘᵢₙ.

For example, when the score Q is defined over a scale between 0 and 1, a representative example of a value for a predetermined score threshold Qₘᵢₙ, is from about 0.5 to about 0.9, e.g., about 0.7.

Representative examples for signal quality parameters that can be used for calculating the score Q include, without limitation, electrode-tissue impedance, proportion of artifacts in the time domain, signal-to-noise ratio in the time domain, parietal alpha-wave power in the frequency domain, frontal theta-wave power effect in the frequency domain, and parietal alpha-wave demanding cognitive tasks power in the frequency domain. In some embodiments of the present invention one or more, optionally and preferably each, of these parameters is subjected to thresholding and the results of the thresholding can be used for calculating the score Q.

A typical threshold for the artifacts in the time domain parameter, when calculated for the overall record duration, is from about 18% to about 22%, e.g., about 20%, wherein a signal for which the value of this parameter is below or equals the threshold is assigned with a score that is higher than a signal for which the value of this parameter is above the threshold.

The signal-to-noise ratio in the time domain parameter can be calculated in units of dB according to the formula SNR=10 log₁₀(σₓ²/σₑ²), where σₓ² and σₑ² are, respectively, the variances of the signal and the noise. A typical threshold for this parameter is from about −2 dB to about 0 dB, e.g., about −1 dB, wherein a signal for which the value of this parameter is above or equals the threshold is assigned with a score that is higher than a signal for which the value of this parameter is below the threshold.

A typical threshold for the parietal alpha-wave power in the frequency domain parameter is from about 3% to about 9%, e.g., about 6%, wherein a signal for which the value of this parameter is above or equals the threshold is assigned with a score that is higher than a signal for which the value of this parameter is below the threshold. A typical threshold for the frontal theta-wave power in the frequency domain parameter is from about 1% to about 5%, e.g., about 3%, wherein a signal for which the value of this parameter is above or equals the threshold is assigned with a score that is higher than a signal for which the value of this parameter is below the threshold. A typical threshold for the parietal alpha-wave demanding cognitive tasks power in the frequency domain parameter is from about 1% to about 5%, e.g., about 3%, wherein a signal for which the value of this parameter is above or equals the threshold is assigned with a score that is higher than a signal for which the value of this parameter is below the threshold.

If the quality is above the quality threshold, the procedure loops back to 111. If, at decision 117, the quality is below the quality threshold, the procedure continues to 118 at which the force is set to zero, for example, by terminating a previously applied force. From 117 the procedure loops back to 111.

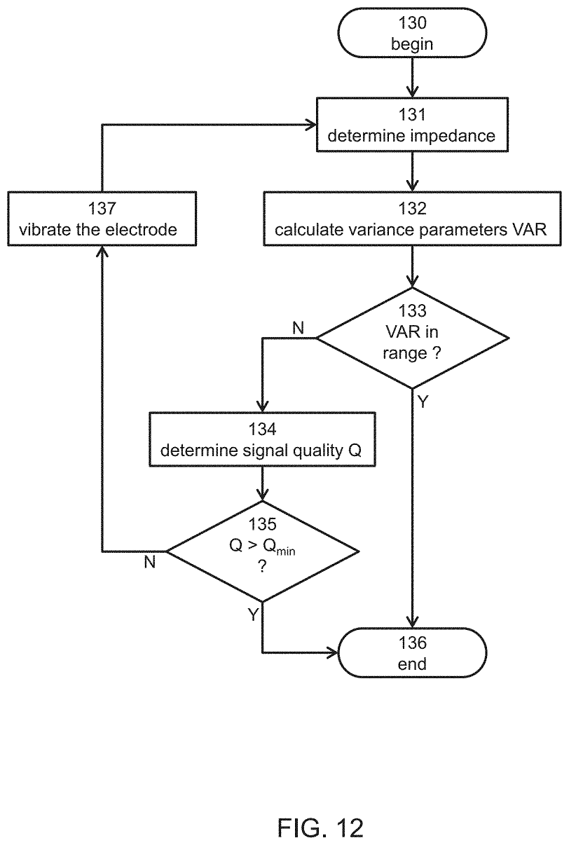

FIG. 12 is a flowchart diagram describing a close loop procedure for operating an actuator to vibrate the electrodes, according to some embodiments of the present invention. The procedure can be executed, for example, by system 10, particularly by actuator 70, electrodes 16, processor 18 and controller 72. The procedure begins at 130 and continues to 131 at which the electrode-tissue impedance is determined, for example, by signal processor 18. The procedure continues to 132 at which variance parameters are calculated, e.g., by signal processor 18, among signals received from a plurality of the electrodes. The procedure continues to decision 133 at which the calculated VAR is compared to a predetermined variance range. Typically the predetermined variance range is chartered by a lower variance limit V_{L} and an upper variance limit V_{U}.

A representative example of impedance value for the lower variance limit V_{L} is from about 5 KΩ to about 100 KΩ, e.g., about 75 KΩ. A representative example of impedance value for the upper variance limit V_{U} is from about 250 KΩ to about 500 KΩ, e.g., about 350 KΩ.

If the calculated VAR is within the range (e.g., V_{U}>VAR>V_{L}), the procedure continues to 136 at which it ends. If the calculated VAR is outside the range (e.g., VAR>V_{U} or VAR<V_{L}), the procedure continues to 134 at which the quality of the signal is determined. This is optionally and preferably done by processor 18 and may include calculating at least one of the power and the frequencies in the signal, signal to noise ratio and artifacts percentage. The procedure continues to decision 135 at which the quality of the signal is compared to a quality threshold or a set of quality thresholds. For example, data processor 18 can calculate a score Q using the calculated quantities and compares the score to a predetermined score threshold Qₘᵢₙ, as further detailed hereinabove. If the quality is above the quality threshold, the procedure continues to 136 at which the procedure ends. If, at decision 135, the quality is below the quality threshold, the procedure continues to 137 at which the actuator is operated to initiate vibration. From 137 the procedure loops back to 131.

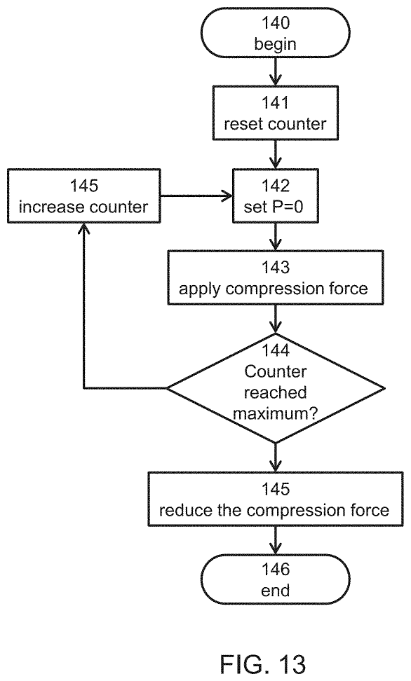

FIG. 13 is a flowchart diagram describing a close loop procedure for operating an actuator to provide a hammering effect, according to some embodiments of the present invention. The procedure can be executed, for example, by system 10, particularly by actuator 70, electrodes 16, processor 18 and controller 72. The procedure begins at 140 and continues to 141 at which a counter is reset. The procedure continues to 142 at which the force applied by the actuator is terminated. If there is no force that is applied at the beginning 140 of the procedure, operation 141 can be skipped. When the actuator comprises a balloon, operation 141 include reducing the pressure in the balloon to the ambient pressure. The procedure continues to 143 at which the actuator is operated to apply compression force at a predetermined magnitude and predetermined profile in the time domain (e.g. ramp, Sine, step, etc.). For example, when the actuator comprises an inflatable balloon, the balloon is inflated to a predetermined pressure. In some embodiments of the present invention the force applied at 143 is the maximal allowed force. At decision 144 the value of the counter is compared to a predetermined maximum count parameter. If the counter does not reach the maximum, the counter is increased 145 by 1, and the procedure loops back to 142. If the counter reaches the maximum, the procedure continues to 146, at which the magnitude of the force is reduced to a predetermined reference level, and to 146 at which the procedure ends.

FIG. 15 is a flowchart diagram describing a combined procedure for operating an actuator, according to some embodiments of the present invention. The procedure can be executed, for example, by system 10, particularly by actuator 70, electrodes 16, processor 18 and controller 72. The procedure begins at 150 and continues to 151 at which a counter is reset. The procedure continues to 113 at which a value of a parameter indicative of the force applied by the actuator is obtained, as further detailed hereinabove. The procedure continues to decision 114 at which the parameter is compared to a predetermined threshold Pₘₐₓ as further detailed hereinabove. If the parameter is below Pₘₐₓ, the procedure continues to 111 at which the electrode-tissue impedance is determined, as further detailed hereinabove. If the parameter is above or equal to Pₘₐₓ, the procedure continues to 152 at which the applied force is reduced.

From 111 the procedure optionally and preferably continues to 115 at which an EEG signal is received from the electrodes and to 116 at which the quality of the signal is determined, as further detailed hereinabove. At decision 153 the quality of the signal is compared to a quality threshold or a set of quality thresholds, and the impedance is compared to a first predetermined threshold Rₘₐₓ, as further detailed hereinabove. If at least one of the quality of the signal (according to one or more of the measures described herein) and the impedance satisfy the respective criterion at 153 the procedure loops back to 113. Otherwise, the procedure continues to 154 at which the electrode is rotated and vibrated, and to 120 at which the force is increased as further detailed hereinabove.

From 154 the procedure loops back to 113.

From 152 the procedure continues to 154 at which the electrode is rotated and vibrated. In some embodiments of the present invention 154 is executed by means of a reciprocal activation of the aforementioned push-to-rotate mechanism embodied as stator 77 and shaft 75. Specifically, force 42 is applied and released repeatedly so that the electrode is rotated in one direction when the force is applied and in the opposite direction when the force is released.

At 155 the counter is increased by 1, and at decision 156 the value of the counter is compared to a predetermined maximum count parameter. If the counter does not reach the maximum, the procedure loops back to 113. If the counter reaches the maximum, the procedure continues to 157, at which the counter is reset and the force is set to zero, as further detailed hereinabove. From 157 the procedure loops back to 113.

Reference is now made to FIGS. 16A-D which are schematic illustrations of a jig system 180 according to some embodiments of the present invention. Jig system 180 is particularly useful for connecting electrode assembly 170 with housing 164 of sensing system 40 to establish electrical communication between legs 46 and circuit board 44. EEG system 10 and jig system 180 can be distributed to users as a kit. The kit can also include one or more sets of disposable electrode assemblies 170 of sensing system 40 each electrode assembly 170 may include body 54 carrying legs 46 as further detailed hereinabove.

Jig system 180 can comprise a scaffold 182 having an outer surface 184 and an inner surface 186. Outer surface 184 is designed and constructed to fittedly receive inner shell 12b of system 10 (not shown in FIGS. 16A-C, see FIGS. 9A-C), and is formed with a plurality of recesses 188 each being size-wise and shape-wise compatible with disposable electrode assembly 170 of sensing system 40.

Jig system 180 also comprises a plurality of jig shafts 190 respectively introduced into the plurality of recesses 188 from the side of inner surface 186 via through holes 192 formed in scaffold 182, in a manner that each jig shaft 190 is able to protrude through a base of the respective recess. In use of jig system 180, a plurality of disposable electrode assemblies 170 of sensing system 40, including bodies 54 with legs 46, are placed in recesses 188, thereby pushing jig shafts 190 inwardly to protrude out of inner surface 186 of scaffold 182 (FIGS. 16B and 16C).

Inner shell 12b of system 10 is then mounted on scaffold 182 (FIG. 16D). Preferably, the shape and size of scaffold 182, and the locations of recesses 188 are selected such that once inner shell 12b is then mounted on the outer surface 184, each one of housings 164 is aligned with a respective recess 188. Shafts 190 are then pushed 194 outwardly in the direction of outer surface 184 to release electrode assemblies 170 out of recesses 188 and to connect electrode assemblies 170 to housings 164 by means of snap members 166.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular from “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

These Examples describe stimulation types and the corresponding cortex area at which an ERP signals is induced.

### Example 1

Electrical Stimulation

Table 1, below, lists limb stimulations and the corresponding cortex locations at which an ERP signal can be induced. Following is a representative example for a stimulation protocol that can be used for limb stimulation.

For upper limb median nerve placement a cathode can be placed between the tendons of palmaris longus and flexor carpi radialis, 2 cm proximal to wrist crease, an anode can be placed from about 2 to about 3 cm distal to the cathode or the dorsum of the wrist, and the ground electrode can be metal plate or circumferential band electrode or a stick on electrode placed on the forearm, or to use the ground electrode of the EEG system.

For lower limb posterior tibial nerve placement, a cathode can be placed between the medial border of the Achilles tendon and the posterior border of the medial malleolus, and an anode can be placed 3 cm distal to the cathode.

The following parameters can be used for the electric stimulation: impedance less than 5 KΩ, a ground electrode on the same limb, monophasic rectangular pulses using constant voltage/constant current stimulator, pulse width from about 100 μs to about 300 μs, stimulation rate of from about 3 Hz to about 5 Hz, analysis time of about 40 ms for median, and about 60 ms for posterior tibial, number of trials from about 500 to about 1000.

TABLE 1

| Limb | Cortex area |
|---|---|
| Left wrist | hand area in right somatosensory cortex (lateral) |
| Right wrist | hand area in left somatosensory cortex (lateral) |
| Left ankle | leg area in right somatosensory cortex (medial) |
| Right leg | leg area in left somatosensory cortex (medial) |

### Example 2

Auditory Stimulation

Table 2, below, lists auditory stimulations and the corresponding cortex locations at which an ERP signal can be induced. Following is a representative example for a stimulation protocol that can be used for auditory stimulation.

The stimulation can be in the form of audio clicks at the ears of the subject, by conformable earplugs connected to a transducer or in-ear speakers or oscillator arranged to vibrate the bones (bone conduction). Auditory stimulation with broad-band clicks is preferred. Click intensity can be about 100 dB pe SPL or from about 60 to about 70 dB HL. Alternating polarity clicks can be used to reduce artifacts. Stimulus rates of from about 5 clicks per second to about 12 clicks per second. The analysis time can be about 15 ms from stimulus onset. The number of trials from about 500 to about 1000. When stimulating the ears sequentially, the non-stimulated ear is optionally and preferably masked with a white noise at 60 dB pe SPL or from about 30 to about 35 dB HL to eliminate “crossover” responses (e.g., bone-conducted responses originating in the non-stimulated ear).

TABLE 2

| Ear | Cortex area |
|---|---|
| Left ear | Left temporal auditory cortex (+pons + midbrain − deep |
| | brainstem) |
| Right ear | Right temporal auditory cortex (+pons + midbrain − deep |
| | brainstem) |

### Example 3

Visual Stimulation

Table 3, below, lists visual stimulations and the corresponding cortex locations at which an ERP signal can be induced. Following is a representative example for a stimulation protocol that can be used for visual stimulation. Visual stimuli can be used at a rate of about 1 Hz. The stimulus can be presented using a photo-stimulator lamp or a LED matrix, or a screen with a known pattern, optionally and preferably with a stroboscope. Preferably, the sound of the stroboscope is masked by white audio noise.

TABLE 3

| Visual Stimulus | Cortex area |
|---|---|
| Flash | the primary visual cortex (occipital lobe) |
| Pattern | calcarine sulcus next to the primary visual cortex |
| | (occipital lobe) |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A system for measuring electroencephalography (EEG) signals, the system comprising:
a wearable body adapted to fit over a scalp;
a plurality of electrodes mounted on said wearable body at a density of at least 2 electrodes per 3 cm²; and
a signal processor configured for detecting Evoked Related Potential (ERP) signals from said electrodes and determining a physiological location of each electrode or each group of electrodes based on said ERP signals.

2. The system according to claim 1, further comprising input for receiving from a stimulation system signals describing stimulation of a subject wearing said wearable body, wherein said signal processor configured for determining said location based in part on said signals from said stimulation system.

3. The system according to claim 2, wherein said stimulation system is configured to apply electrical stimulation by at least one of said electrodes.

4. (canceled)

5. The system according to claim 1, comprising a plurality of physically separate sensing systems, each comprising several of said plurality of electrodes.

6. A system for measuring electroencephalography (EEG) signals, the system comprising:
a wearable body adapted to fit over a scalp;
a plurality of electrodes mounted on said wearable body;
a plurality of controllable actuators for applying force to said electrodes;
a controller configured for individually controlling each actuator or group of actuators to apply force to at least one electrode; and
a signal processor configured for receiving and processing signals from said electrodes and transmitting control signals to said controller based on said processing.

7. The system of claim 6, wherein said signal processor is configured for determining at least one of: an electrode-tissue impedance, a signal-to-noise ratio, artifacts percentage, a signal quality, and actuating pressure, and to control said force based on said determination.

8. The system according to claim 6, wherein said force is applied inwardly.

9. The system according to claim 8, wherein at least one of said electrodes is flexible and configured to experience a strain once pressed by said force against said scalp.

10. The system according to claim 6, wherein said actuator is configured to apply said force while establishing rotary motion to said electrodes.

11. (canceled)

12. The system according to claim 6, wherein at least one of said controllable actuators comprises an inflatable balloon or a pneumatic actuator, applying said force upon inflation thereof.

13. (canceled)

14. The system according to claim 6, wherein said force is periodic and is applied to vibrate said electrodes or generate a hammering effect.

15. (canceled)

16. The system according to claim 6, comprising a plurality of physically separate sensing systems, each comprising several of said plurality of electrodes.

17. (canceled)

18. The system according to claim 16, wherein at least one of said sensing systems comprises a circuit board and a plurality of flexible legs, each having a non-conductive section and a conductive section having a tip in electrical communication with said circuit board, and wherein each conductive section is one of said plurality of electrodes.

19. (canceled)

20. The system according to claim 18, wherein said circuit board and said plurality of flexible legs are detachable from each other.

21. (canceled)

22. The system according to claim 18, wherein said wearable body comprises an inner shell supporting said circuit board and said plurality of flexible legs, and an outer shell supporting said plurality of controllable actuators.

23. (canceled)

24. The system according to claim 18, wherein at least one of said flexible legs has a helical shape.

25. (canceled)

26. The system according to claim 18, wherein a conductive section of at least one of said plurality of legs is polymeric.

27. (canceled)

28. The system according to claim 18, wherein a conductive section of at least one of said plurality of legs comprises a bundle of conductive bristles.

29. (canceled)

30. The system according to claim 18, comprising a controllable vibrating member configured for vibrating said legs.

31. (canceled)

32. The system according to claim 18, wherein at least one of said legs comprises a hydrophobic zone at an upper part of said leg and a hydrophilic zone at a lower part of said leg.

33. (canceled)

34. The system according to claim 32, wherein said at least one of said legs comprises an intermediate zone between said hydrophobic zone and said hydrophilic zone, said intermediate zone being less hydrophobic than said hydrophobic zone, and less hydrophilic than said hydrophilic zone.

35. (canceled)

36. The system according to claim 18, wherein said plurality of flexible legs is arranged on a base of a sensing system body, wherein said at least one sensing system comprises a shaft and a housing mounted on said shaft and being configured to receive said sensing system body, and wherein said housing comprises a rigid wall for holding said sensing system body and a flexible membrane connecting said rigid wall with said shaft in a manner that allows said housing to assume a plurality of different orientations with respect to said shaft.

37. (canceled)

38. The system according to claim 1, comprising a controller for controlling a connection state of each individual electrode.

39-41. (canceled)

42. The system according to claim 38, wherein said controller is configured for electrically grouping said electrodes into at least one group.

43-47. (canceled)

48. A method of measuring electroencephalography (EEG) signals, the method comprising operating the system according to claim 1, while said wearable body is placed on a scalp of a subject, to receive EEG signals sensed by said plurality of electrodes, thereby measuring the EEG signals.

49-50. (canceled)

51. A system for sensing electroencephalography signals, comprising a circuit board and a plurality of flexible legs, each having a non-conductive section and a conductive section having a tip in electrical communication with said circuit board, wherein a conductive section of at least one of said plurality of legs comprises a bundle of conductive bristles.

52-55. (canceled)

56. A method of measuring electroencephalography (EEG) signals, the method comprising:
receiving signals from a plurality of electrodes placed on a surface scalp of a subject; and
processing said signals and controlling a plurality of controllable actuators to apply force to said electrodes, based on said processing.

57. The method of claim 56, comprising determining at least one quantity selected from the group consisting of: an electrode-tissue impedance, a signal-to-noise ratio, an artifacts percentage, signal quality, and pneumatic pressure, and increasing said force responsively to a value of said at least one quantity.

58-75. (canceled)

76. A jig system for collectively assembling a plurality of sensing systems, each sensing system being configured for sensing electroencephalography (EEG) signals, the jig system comprising:
a scaffold having an outer surface and an inner surface, said outer surface being designed and constructed to fittedly receive a wearable body mounted with a plurality of housings;
a plurality of recesses formed in said outer surface, each having a base and a through hole formed between said base and said inner surface of said scaffold, each recess being size-wise and shape-wise compatible with a disposable electrode assembly of one of the sensing systems; and
a plurality of jig shafts, each introduced into one of said recesses from a side of said inner surface via a respective through holes.

77. A kit, comprising:
the jig system of claim 76; and
a system for measuring electroencephalography (EEG) signals, the system comprising a wearable body adapted to fit over a scalp, and a plurality of housings mounted on said wearable body, each housing having therein a circuit board of one of the sensing systems, and being configured to receive an electrode assembly of one of the sensing systems.

78. The kit according to claim 77, comprising a plurality of disposable electrode assemblies each being size-wise and shape-wise compatible with one of said recesses.

79. (canceled)
